(19) United States
(12) Patent Application Publication
(10) Pub. No.: US 2012/0264231 A1
(43) Pub. Date: Oct. 18, 2012

(54) ASSAY FOR ORAI CALCIUM CHANNEL REGULATORS

(75) Inventors: Patrick Hogan, Cambridge, MA (US); Yubin Zhou, San Diego, CA (US); Anjana Rao, Cambridge, MA (US); Paul Meraner, San Diego, CA (US); Danya Bess Machnes, Boston, MA (US)

(73) Assignee: IMMUNE DISEASE INSTITUTE, INC., Boston, MA (US)

(21) Appl. No.: 13/379,326

(22) PCT Filed: Jun. 21, 2010
(86) PCT No.: PCT/US10/39340

§ 371 Date: Jun. 8, 2012

Related U.S. Application Data
(60) Provisional application No. 61/218,783, filed on Jun. 19, 2009.

(51) Int. Cl. G01N 33/566; C12N 1/19; G01N 21/64

(52) U.S. Cl. 436/501; 435/254.21; 435/254.23

(57) ABSTRACT

The methods and systems described herein are based, in part, on the discovery that STIM modulates calcium release from store-operated channels through a direct interaction with the ORAI channel. Based on this discovery, methods and systems are described herein for identifying an agent that modulates calcium flux through the ORAI channel and/or regulates intracellular calcium via the ORAI channel. The methods and systems can also be used to detect an interaction between STIM and a functional ORAI channel.

### CROSS REFERENCE

This Application claims the benefit of priority under 35 U.S.C. §119(e) of U.S. Provisional Application No. 61/218,783, filed Jun. 19, 2009, the contents of which are incorporated herein in their entirety.

### GOVERNMENT SUPPORT

This invention was made with Government support under AI40127 and GM075256 awarded by the National Institutes of Health. The Government has certain rights in the invention.

### SEQUENCE LISTING

The instant application contains a Sequence Listing which has been submitted via EFS-Web and is hereby incorporated by reference in its entirety. Said ASCII copy, created on Jun. 18, 2010, is named 33393652.txt and is 2,552 bytes in size.

### FIELD OF THE INVENTION

The field of the invention relates to the expression of ORAI calcium channel proteins in yeast systems, and assays for regulators performed in such systems.

### BACKGROUND OF THE INVENTION

Influx of Ca²⁺ through the CRAC channel of T cells and mast cells, a classical instance of store-operated Ca²⁺ entry (Parekh, A. B. & Putney, J. W., Jr. Physiol Rev 85, 757-810 (2005); Putney, J. W., Jr. Cell Calcium 42, 103-110 (2007); Hogan, PG & Rao, A. Trends Biochem Sci 32, 235-245 (2007)), requires the proteins STIM1 and ORAI1. The early steps of STIM1-ORAI signaling have been elegantly worked out in studies using engineered fluorescent STIM proteins. STIM1 senses a reduction of ER luminal Ca²⁺ concentration through dissociation of Ca²⁺ from a luminal EF-hand, leading to oligomerization of STIM, and then a local redistribution within the ER by which STIM becomes enriched at sites of ER-plasma membrane apposition, termed puncta (Liou, J., et al., Curr Biol 15, 1235-1241 (2005); Zhang, S. L., et al., Nature 437, 902-905 (2005); Spassova, M. A., et al., Proc Natl Acad Sci USA 103, 4040-4045 (2006); Mercer, J. C., et al., J Biol Chem 281, 24979-24990 (2006); Wu, M. M., et al., J Cell Biol 174, 803-813 (2006); Baba, Y., et al., Proc Natl Acad Sci USA 103, 16704-16709 (2006); Liou, J., et al., Proc Natl Acad Sci USA 104, 930 1-9306 (2007); Ong, H. L., et al., J Biol Chem 282, 121 76-12185 (2007)). Subsequently, STIM1 recruits ORAI1 to ER-plasma membrane contacts, where Ca²⁺ enters the cell through opened ORAI channels (Xu, P., et al., Biochem Biophys Res Comm 350, 969-976 (2006); Luik, R. M., et al., J Cell Biol 174, 815-825 (2006); Luik, R. M., et al., Nature 454, 538-542 (2008); Muik, M., et al., et al., J Biol Chem 283, 8014-8022 (2008); Navarro-Borelly, L., et al., J Physiol 586, 5383-5401 (2008)). Structural and biochemical studies with recombinant ER-luminal portions of STIM 1 and STIM2 have illuminated the molecular mechanism by which STIM proteins sense Ca²⁺ changes in the ER lumen (Stathopulos, P. B., et al., J Biol Chem 281, 35855-35862 (2006); Stathopulos, P. B., et al., Cell 135, 110-122 (2008)).

Despite these insights, it remains unclear whether STIM directly gates ORAI channels. RNAi screens have identified other proteins that contribute significantly to store-operated Ca²⁺ entry (Zhang, S. L., et al., Proc Natl Acad Sci USA 103, 9357-9362 (2006); Vig, M., et al., Science 312, 1220-1223 (2006), suggesting that proteins in addition to STIM and ORAI could have a direct role in channel opening. The observation that overexpression of STIM 1 with ORAI is sufficient for large store-operated Ca²⁺ currents (Zhang, S. L., et al., (2006), supra; Mercer, J. C., et al., J Biol Chem 281, 24979-24990 (2006); Peinelt, C., et al., Nat Cell Biol 8, 771-773 (2006); Soboloff, J., et al., J Biol Chem 281, 20661-20665 (2006)) has been taken as an indication that STIM by itself can gate ORAI. However, the cells used for expression of STIM and ORAI normally possess a store-operated Ca²⁺ entry pathway and thus can be presumed to have the full complement of proteins necessary for store-operated Ca²⁺ entry, and overexpressed ORAI appears to be part of a larger channel complex (Várnai, P., et al., J Biol Chem 282, 29678-29690 (2007)), leaving open the possibility that other proteins in the complex have a necessary role in channel opening.

### SUMMARY OF THE INVENTION

Described herein are methods and systems that relate to detection of an interaction between STIM and ORAI, and/or detection of store-operated calcium release. The methods and systems described herein can be used to identify a candidate agent that modulates calcium flux through the ORAI channel. The system can use cells that lack endogenous STIM and ORAI signaling to reduce background noise levels. Also described herein are compositions for use with the methods and systems described herein.

In one aspect the methods described herein relate to a method of identifying an agent that modulates Ca²⁺ flux through the ORAI channel, comprising: a) providing S. cerevisiae secretory vesicles functionally expressing ORAI or a functional fragment or derivative thereof; b) contacting the secretory vesicles with STIM1, or a functional fragment or derivative thereof, and a test agent; and c) monitoring calcium release from the vesicles; wherein a significant difference in the calcium release from the vesicles compared to a control which lacks the test agent, indicates the test agent modulates Ca²⁺ flux through the ORAI channel.

In one embodiment of this method, and all other methods and compositions described herein, the method is performed in the absence of other mammalian proteins.

In another embodiment of this method, and all other methods and compositions described herein, the S. cerevisiae is a sec 6-4 strain.

In one embodiment of this method, and all other methods and compositions described herein, the method further comprises contacting the vesicles with another mammalian protein known to modulate STIM1 regulation of intracellular calcium.

In one embodiment of this method, and all other methods and compositions described herein, the monitoring step c) is with a calcium detection agent.

In one embodiment of this method, and all other methods and compositions described herein, the calcium detection agent is a fluorescent dye or FRET pairs of GFP variants sensitive to Ca⁺⁺ binding.

In one embodiment of this method, and all other methods and compositions described herein, the fluorescent dye or FRET pair is Fura-2, CFP and/or YFP.

In one embodiment of this method, and all other methods and compositions described herein, the test agent is known to modulate intracellular calcium.

Another aspect of the present invention relates to a method of identifying an agent that modulates ORAI regulation of intracellular calcium, comprising: a) providing yeast secretory vesicles or liposomes expressing a functional ORAI calcium channel; b) contacting the secretory vesicles or liposomes with a test agent; c) monitoring calcium release from the vesicles or liposomes; wherein a significant difference in the calcium release from the vesicles or liposomes compared to a control which lacks the test agent, indicates the test agent modulates ORAI regulation of intracellular calcium.

In one embodiment of this method, and all other methods and compositions described herein, the contacting step b) further comprises contacting the secretory vesicles or liposomes with STIM1 or a functional fragment or derivative thereof.

In one embodiment of this method, and all other methods and compositions described herein, the STIM1 functional fragment is STIM1 (233-685), STIM1 (233-498), STIM1 (233-463), or STIM1 (233-600), and the ORAI functional fragment is ORAI1 (65-301), ORAI1 (65-87), or full length ORAI1.

In one embodiment of this method, and all other methods and compositions described herein, the ORAI or functional fragment thereof comprises an epitope tag.

Aspects of the invention also relate to a system for detection of calcium release consisting essentially of: a) a recombinant ORAI channel functionally expressed, and b) STIM1, functional fragment or derivative thereof, and the use of the system in the methods described herein.

Aspects of the invention also relate to a system comprising: a) a recombinant ORAI protein or fragment or derivative thereof, expressed in yeast or a vesicle or membrane isolated therefrom, and the use of the system in the methods described herein.

In one embodiment of this and other systems, and other methods described herein, the system further comprises STIM1 protein, or a fragment or derivative thereof.

In one embodiment of this and other systems, and other methods described herein, the yeast is S. cerevisiae or P. pastoris.

In one embodiment of this and other systems, and other methods described herein, the ORAI protein or fragment or derivative thereof, and/or the STIM1 protein, or fragment or derivative thereof, is functional.

In one embodiment of this and other systems, and other methods described herein, the system further comprises a calcium detection agent.

In one embodiment of this and other systems, and other methods described herein, the STIM1 protein, or fragment or derivative thereof, is STIM1 (233-685) STIM1 (233-498), STIM1 (233-463), or STIM1 (233-600), and wherein the ORAI protein or fragment or derivative thereof, is ORAI1 (65-301), ORAI1 (65-87), or full length ORAI1.

In one embodiment of this and other systems, and other methods described herein, the system further comprises another mammalian protein or factor.

In one embodiment of this and other systems, and other methods described herein, the system does not comprise another mammalian protein or factor.

Aspects of the invention also relate to a yeast organism, or microsomal membrane or vesicle thereof, that comprises a recombinant, expressed ORAI protein or fragment or derivative thereof, and the use of the organism in the compositions and methods described herein.

In one embodiment of this and other compositions and methods described herein, the yeast organism is genetically engineered to contain, in expressible form, a nucleic acid encoding the ORAI protein or fragment or derivative thereof.

In one embodiment of this and other compositions and methods described herein, the yeast organism comprises S. cerevisiae, comprising a recombinant functionally expressed ORAI channel.

In one embodiment of this and other compositions and methods described herein, the yeast organism further comprises a recombinant functionally expressed calcium sensor.

In one embodiment of this and other compositions and methods described herein, the S. cerevisiae, or isolated vesicle thereof, comprises a sec6-4 strain.

In one embodiment of this and other compositions and methods described herein, the S. cerevisiae, further comprises another mammalian protein known to modulate STIM1 regulation of intracellular calcium.

Aspects of the invention also relate to a yeast organism, or microsomal membranes thereof, that is Pichia pastoris, and the use of the organism or membranes in the methods and compositions described herein.

Another aspect of the invention relates to a method of identifying an agent that modulates STIM1 binding of a functional ORAI channel, comprising: a) providing microsomal membranes prepared from P. Pastoris expressing ORAI or a functional fragment or derivative thereof; and b) performing a membrane flotation assay for binding of STIM1 or a fragment or derivative thereof, to the ORAI, in the presence and absence of a test agent; wherein modulation of binding of STIM1 to the ORAI in the presence of the test agent, compared to binding in the absence of the test agent, indicates that the agent modulates STIM1 binding of a functional ORAI channel.

Another aspect of the invention relates to a method of identifying an agent that modulates STIM1 binding of a functional ORAI channel, comprising: a) providing membranes with ORAI or a fragment or derivative thereof incorporated or reconstituted therein; and b) performing a binding assay for STIM1 or a fragment or derivative thereof, to the ORAI in the membrane, in the presence and absence of a test agent; wherein modulation of binding of STIM1 to the ORAI in the presence of the test agent, compared to binding in the absence of the test agent, indicates that the agent modulates STIM1 binding of a functional ORAI channel.

In one embodiment of this and other methods, and related compositions described herein, the ORAI is ORAI1.

In one embodiment of this and other methods, and related compositions described herein, the ORAL is a functional fragment.

In one embodiment of this and other methods, and related compositions described herein, the STIM1 fragment or derivative thereof is a functional fragment.

In one embodiment of this and other methods, and related compositions described herein, the STIM1 is STIM1 (233-685) or STIM (233-498).

In one embodiment of this and other methods, and related compositions described herein, the performing step b) is in the presence or absence of one or more mammalian proteins known to modulate STIM1 regulation of intracellular calcium.

### BRIEF DESCRIPTION OF THE DRAWINGS

FIGS. 1A-1D show recombinant ORAI1 and recombinant STIM 1 cytoplasmic fragments used herein in the Examples section. FIG. 1A is a schematic, showing how ORAI 1 is incorporated normally into the plasma membrane of sec6-4 yeast at the permissive temperature, 25° C., but accumulates in vesicles within the cell at the non-permissive temperature, 37° C. The expanded view represents ORAI1 orientation in sec6-4 vesicles, with the cytoplasmic portions of ORAI1 facing the external solution. The incorporation and accumulation of ORAI1 was seen by immunocytochemistry of Myc-ORAI1 in sec6-4 cells at 25° C. and at 37° C. (data not shown). FIG. 1B (above) is a representation of the sequence conservation in the STIM C-terminal region. Each horizontal black bar represents the human STIM1 sequence, with gaps introduced as necessary to maintain alignment with fish STIM1 orthologues or with insect Stim proteins, as indicated. Vertical lines indicate identity of the human STIM1 residue with residues at the corresponding position in at least four of five fish orthologues; vertical lines indicate identity with at least two of three residues in insect Stim proteins. Accession numbers are listed herein in the Examples section. FIG. 1B (below) is a drawing representing the recombinant STIM 1 cytoplasmic fragments used herein. Predicted coiled coil (CC), SP-rich (SP), and polybasic (K) regions are indicated. FIG. 1C is a graphical representation of data of SEC-MALLS analysis of STIM1^{CT}. The data indicates that recombinant STIM1^{CT} migrated as a single symmetrical peak on size exclusion chromatography, with no evidence of aggregated protein in the void volume at ˜5-8 mL. Molecular mass estimated from a series of MALLS measurements across each protein peek are plotted, referred to the axis on the right of the panel. STIM 1^{CT} MALLS experimental MW, 110.5 kDa; theoretical monomer MW, 54.7 kDa. Standards were bovine serum albumin (BSA, black), experimental MW, monomer 69.6 kDa, dimer 134.8 kDa; theoretical monomer MW, 67.0 kDa; and dimeric S. japonicum glutathione S-transferase (GST, gray) experimental MW, 43.4 kDa; theoretical dimer MW, 50.0 kDa. STIM^{CT} elutes earlier than expected for a globular protein of the same MW. FIG. 1D is a graphical representation of data. Molecular masses estimated from a series of MALLS measurements across selected protein peaks are plotted, referred to the axis on the right of the panel. STIM1 (233-498) experimental MW, 70.1 kDa; theoretical monomer MW, 34.8 kDa; STIM 1 (233-463) experimental MW, 87.5 and 119.6 kDa; theoretical monomer MW, 30.9 kDa. Heterogeneity of STIM 1 (233-463) is evident in the asymmetric trimer peak, a small tetramer peak, and a substantial amount of large aggregates. Material eluting in the void volume and in the trailing edge of the trimer peak was of indeterminate MW.

FIGS. 2A-2C show experimental results that indicate that STIM 1 cytoplasmic fragments interact with ORAI1 assembled in yeast membranes and with the recombinant C-terminal cytoplasmic tail of ORAI 1. FIG. 2A (above) is a drawing that highlights the part of ORAI (ORAI1 (65-301)) that was expressed as a membrane protein in P pastoris. FIG. 2A (below) shows the results of experiments in which Pichia membranes containing FLAG-ORAI1 (65-301) or control membranes, with or without His6-STIM1 protein, were loaded at the bottom of a discontinuous sucrose density gradient and subjected to centrifugation. FLAG-ORAI and His6-STIM in individual gradient fractions were detected by Western blotting. The fraction of unbound STIM remaining at the bottom of the gradient was likely due to the presence of a moderate excess of STIM over ORAI in the assay. FIG. 2B (above) is a drawing that highlights the C-terminal segment of ORAI (ORAI1 (259-301)) that was expressed as a GST fusion protein. FIG. 2B (below) shows the results of experiments in which the indicated STIM fragments were incubated with immobilized GST-ORAI1 (259-301) or with GST. Bound proteins were analyzed by SDS-PAGE and staining with Coomassie Brilliant Blue R-250. Samples on the input gel correspond to 20% of protein in the binding assay. FIG. 2C (above) is a drawing that highlights the segment of ORAI (ORAI1 (65-87)) that was expressed as a GST fusion protein. FIG. 2C (below) shows the results of experiments in which the indicated STIM fragments were incubated with immobilized GST-ORAI1 (65-87) or with GST and analyzed as in FIG. 2C. Samples on the input gel correspond to 5% of protein in the binding assay.

FIGS. 3A-3B show experimental results that indicate that STIM 1 triggers ORAI-dependent Ca2+ efflux from membrane vesicles of S. cerevisiae. FIG. 3A (top panel) is a schematic that shows the principle of the Ca²⁺ flux assay using Fura-2. An increase in the effectiveness of exciting light at 340 nm corresponds to an increase in extravesicular Ca²⁺. FIG. 3A (middle panels) are graphical representations of Fura-2 excitation spectra of control and ORAI1-containing vesicles, with no addition or after the addition of ionomycin (20 μM) or STIM 1 cytoplasmic fragments (2 μM). FIG. 3A (lower panel) is a bar graph of the fluorescence intensity ratio of Fura-2 (F340 nm/F380 nm) in each condition. Error bars indicate the range of duplicate measurements.

FIG. 3B (top panel) is a schematic that shows the principle of the Ca²⁺ flux assay using the FRET-based Ca²⁺ sensor cameleon D4 cpV. A decrease in fluorescence emission at 528 nm and increase in emission at 475 nm corresponds to a decrease in vesicular Ca²⁺. FIG. 3A (middle panels) are graphical representations of D4 cpV fluorescence emission spectra of control and ORAI1-containing vesicles, with no addition or after the addition of ionomycin (20 μM) or STIM 1 cytoplasmic fragments (2 μM). FIG. 3A (lower panel) is a bar graph of the fluorescence intensity ratio of D4 cpV (F528 nm/F475 nm) in each condition. Error bars indicate the range of duplicate measurements.

FIG. 4A-FIG. 4D shows data indicating that STIM 1 activates ORAI1 channels in membrane vesicles from S. cerevisiae. The experiment was carried out as in FIG. 3B except for the use of D3 cpV sensor. FIGS. 4A-4C are graphical representations of fluorescence emission spectra obtained from vesicles containing (FIG. 4A) wildtype ORAI, (FIG. 4B) ORAI(R91W), or (FIG. 4C) ORAI(E106Q), either with no addition or following addition of STIM^{CT} (2.6 μM). FIG. 4D is a bar graph that plots mean fluorescence intensity ratio F528 nm/F475 nm±s.e.m. for each condition. STIM1^{CT} changes the ratio significantly for wildtype ORAI1 (p<0.001, two-tailed Welch's t test), but not for ORAI1 (R91W) and ORAI1 (E106Q).

FIG. 5 is a drawing showing a model for STIM-ORAI signaling in cells. When activated STIM1 oligomers collect at ER-plasma membrane contacts, (1) the STIM 1 coiled coil bridges the distance separating ER and plasma membrane; (2) STIM 1 recruits ORAI1 through a direct interaction with the C terminus of one or more ORAI1 channel subunits; and (3) STIM1 opens ORAI1 channels through a further direct protein-protein interaction. STIM 1 is depicted here as a dimer, the species present in the work described herein with soluble STIM 1^{CT}, but STIM as a dimer or a higher oligomer at puncta is also contemplated herein.

FIG. 6 is a diagram of full-length STIM1. The cytoplasmic region contains three predicted coiled-coil regions, an SP-rich region, and a polybasic tail. The first predicted coiled coil spans a distance approximately equal to the distance, ˜17 nm (Wu, M. M., et al., J Cell Biol 174, 803-813 (2006)), separating ER and plasma membrane at the close appositions where STIM and ORAI accumulate upon ER Ca²⁺ store depletion. Coiled coil 1 and coiled coil 2 have long been recognized in STIM proteins (Oritani, K. P. & Kincade, W. J Cell Biol 134, 771-782 (1996); Manji, S. S. M., et al., Biochim Biophys Acta 1481, 147-155 (2000); Williams, R. T., et al., Biochem J 357, 673-685 (2001)) and are assigned high probability in STIM 1 by COILS (Lupas, A., et al., Science 252, 1162-1164 (1991)). The potential coiled coil 3 is assigned a lower probability in STIM1 but a relatively high probability in some STIM homologues.

FIG. 7 contains graphical representations showing the far-UV CD spectrum of STIM1CT indicating a structured protein with 49% α-helix. The left inset shows the thermal unfolding curve has a sharp melting transition (Tm: 47.0±0.3° C.) consistent with the presence of a single folded species. The right inset shows a photo of SDS-PAGE analysis demonstrates homogeneity of the purified protein.

FIG. 8 contains graphical representations showing the far-UV CD spectra of STIM1 (233-498) and STIM1 (233-463) indicating 54% and 57% α-helix, respectively. The left inset shows the thermal unfolding curves (Tm: 48.5±0.4° C. and 43.8±0.2° C., respectively). The right inset is a photo of SDS-PAGE analysis of the purified proteins.

FIG. 9 is a schematic depicting the principle of the Ca²⁺ flux assay using Fura-271. Fura-2 is effectively the sole Ca²⁺ buffer in the solution surrounding the vesicles. Initially, the external Ca²⁺ concentration is low and the peak of the free Fura-2 excitation spectrum is ˜365 nm. When STIM 1 gates the ORAI1 channel, Ca²⁺ is released from the vesicles, Fura-2 binds Ca²⁺, and the excitation peak is shifted to ˜340 nm.

FIG. 10 is a schematic depicting the principle of the Ca²⁺ flux assay using the FRET-based Ca²⁺ sensors cameleon D3 cpV and D4 cpV72. In unstimulated vesicles, internal Ca²⁺ concentration is sufficient for binding to a fraction of the sensor, and CFP-YFP FRET is evident in the peak at ˜528 nm. When STIM1 gates the ORAI1 channel, Ca²⁺ is released from the vesicles, Ca²⁺ dissociates from the sensor, and there is a decline in the YFP peak at ˜528 nm accompanied by an increase in the CFP peak at ˜475 nm.

FIG. 11 is a series of charts showing expression of STIM1 cytoplasmic fragments in STIM1^{-/-} T cells causes constitutive Ca2+ influx. FIG. 11A is a graph of results from an experiment whereby cytoplasmic [Ca²⁺] monitored by Fura-2 in STIM1^{-/-} T cells reconstituted with the indicated STIM protein or with empty vector. The rapid, reversible elevation in [Ca²⁺] upon exposure to external Ca²⁺ is diagnostic of elevated resting Ca²⁺ permeability. The rapid decline upon exposure to La³⁺, in the continuing presence of external Ca²⁺, further confirms that STIM1 proteins cause an ongoing Ca²⁺ influx. Retroviral vector alone and full-length STIM1, which requires Ca²⁺ store depletion for activation, serve as negative controls. FIG. 11B is a set of bar graphs which represent the peak cytoplasmic [Ca²⁺] and the maximal rate of change in [Ca²⁺] averaged from two independent experiments (n=50-100 cells in each condition in each experiment). Error bars indicate the range of the means of the individual experiments.

FIG. 12 is a graphical representation of spectra obtained from control membranes and membranes containing ORAI1 (E106Q), taken before and after the addition of Tb3⁺.

### DETAILED DESCRIPTION

### Definitions

The term “fragment” or “derivative” when referring to a STIM1 or ORAI protein means proteins or polypeptides which share at least some amino acid sequence of the native full length protein. In one embodiment, the fragment or derivative retains essentially the same biological function or activity in at least one assay as the native full length protein. One such activity for STIM1 is binding to ORAI1. Another such activity is activation of ORAI1. In one embodiment, the fragment or derivative of the present invention maintain at least about 50% of the retained activity of the native protein, preferably at least 75%, more preferably at least about 95% of the activity of the native proteins, as determined e.g., by binding assay or by a calcium influx assay, such as that described in WO 2007/081804.

A fragment of a sequence contains less nucleotides or amino acids than the corresponding full length sequences, wherein the sequences present are in the same consecutive order as is present in the full length sequence. As such, a fragment does not contain internal insertions or deletions of anything (e.g. nucleic acids or amino acids) in to the portion of the full length sequence represented by the fragment. This is in contrast to a derivative, which may contain internal insertions or deletions within the nucleic acids or amino acids that correspond to the full length sequence, or may have similarity to full length coding sequences. A fragment may be considered as functional or non-functional. Functional will depend upon the specific protein which the term describes. Functional STIM1 refers to having the ability to bind to and/or activate the ORAI channel as determined by any number of methods known in the art (e.g., can reconstitute store-operated Calcium influx or CRAC current in STIM1^{-/-} cells, or can activate ORAI calcium channel function in the vesicle assay described herein).

A derivative may comprise the same or different number of nucleic acids or amino acids as full length sequences. The term derivative, as used herein includes proteins, or fragments thereof, which contain one or more modified amino acids. e.g. chemically modified, or modification to the amino acid sequence (substitution, deletion, or insertion). One example of a modification to the amino acid sequence is a conservative substitution mutation, described below. Modifications which create derivatives can substantially preserve a desired activity of the protein (e.g., the ORAI modulatory and/or binding activity of STIM1, the calcium channel function of the ORAI protein, the membrane integration of the ORAI protein, etc.). Such activity is readily determined by a number of assays known in the art, for example, a binding assay, or a calcium influx assay can be used to determine calcium channel function. By way or nonlimiting example, a derivative may be prepared by standard modifications of the side groups of one or more amino acid residues of the protein, its analog, or a functional fragment thereof, or by conjugation of the protein, its analogs or fragments, to another molecule e.g. an antibody, enzyme, receptor, etc., as are well known in the art. Accordingly, “derivatives” as used herein covers derivatives which may be prepared from the functional groups which occur as side chains on the residues or the N- or C-terminal groups, by means known in the art, and are included in the invention. Derivatives may have chemical moieties such as carbohydrate or phosphate residues. Derivatives can be made for convenience in expression, for convenience in a specific assay, to enhance detection, or for other experimental purposes. Derivatives include dominant negatives, dominant positives and fusion proteins. In one embodiment, a derivative has at least 90% amino acid sequence identity to the native protein, or a functional fragment of the native protein.

As well-known in the art, a “conservative substitution” of an amino acid or a “conservative substitution variant” of a polypeptide refers to an amino acid substitution which maintains: 1) the structure of the backbone of the polypeptide (e.g. a beta sheet or alpha-helical structure); 2) the charge or hydrophobicity of the amino acid; or 3) the bulkiness of the side chain. More specifically, the well-known terminologies “hydrophilic residues” relate to serine or threonine. “Hydrophobic residues” refer to leucine, isoleucine, phenylalanine, valine or alanine. “Positively charged residues” relate to lysine, arginine or histidine. “Negatively charged residues” refer to aspartic acid or glutamic acid. Residues having “bulky side chains” refer to phenylalanine, tryptophan or tyrosine.

Conservative amino acid substitutions are well understood in the art, and relate to substitution of a particular amino acid by one having a similar characteristic (e.g., similar charge or hydrophobicity, similar bulkiness). Examples include aspartic acid for glutamic acid, or isoleucine for leucine. A list of exemplary conservative amino acid substitutions is given in the table below. A conservative substitution mutant or variant will 1) have only conservative amino acid substitutions relative to the parent sequence, 2) will have at least 90% sequence identity with respect to the parent sequence, preferably at least 95% identity, 96% identity, 97% identity, 98% identity or 99% or greater identity; and 3) will retain protein activity as that term is defined herein.

CONSERVATIVE AMINO ACID REPLACEMENTS

| For | | |
|---|---|---|
| Amino Acid | Code | Replace With |
| Alanine | A | D-ala, Gly, Aib, β-Ala, Acp, L-Cys, D-Cys |
| Arginine | R | D-Arg, Lys, D-Lys, homo-Arg, D-homo-Arg, |
| | | Met, Ile, D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S—Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, Aib, β-Ala, Acp |
| Isoleucine | I | D-Ile, Val, D-Val, AdaA, AdaG, Leu, D-Leu, |
| | | Met, D-Met |
| Leucine | L | D-Leu, Val, D-Val, AdaA, AdaG, Leu, D-Leu, |
| | | Met, D-Met |
| Lysine | K | D-Lys, Arg, D-Arg, homo-Arg, D-homo-Arg, |
| | | Met, D-Met, Ile, D-Ile, Orn, D-Orn |
| Methionine | M | D-Met, S—Me-Cys, Ile, D-Ile, Leu, D-Leu, |
| | | Val, D-Val |
| Phenylalanine | F | D-Phe, Tyr, D-Thr, L-Dopa, His, D-His, |
| | | Trp, D-Trp, Trans-3,4 or 5-phenylproline, AdaA, |
| | | AdaG, cis-3,4 or 5-phenylproline, Bpa, D-Bpa |
| Proline | P | D-Pro, L-I-thioazolidine-4-carboxylic acid, |
| | | D-or-L-1-oxazolidine-4-carboxylic acid (Kauer, |
| | | U.S. Pat. No. (4,511,390) |
| Serine | S | D-Ser, Thr, D-Thr, allo-Thr, Met, D-Met, |
| | | Met (O), D-Met (O), L-Cys, D-Cys |
| Threonine | T | D-Thr, Ser, D-Ser, allo-Thr, Met, D-Met, |
| | | Met (O), D-Met (O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met, |
| | | AdaA, AdaG |

The term “agent” or “compound” as used herein and throughout the specification means any organic or inorganic molecule, including modified and unmodified nucleic acids such as antisense nucleic acids, RNAi, such as siRNA or shRNA, nucleic acid analogues, proteins, polypeptides, protein or polypeptide inhibitors, peptidomimetics, chemicals, small molecules, chemical entities, receptors, ligands, and antibodies. A protein and/or peptide inhibitor or fragment thereof, can be, for example, but not limited to mutated proteins; therapeutic proteins and recombinant proteins. Protein and peptide inhibitors can also include for example; mutated proteins, genetically modified proteins, peptides, synthetic peptides, recombinant proteins, chimeric proteins, antibodies, humanized proteins, humanized antibodies, chimeric antibodies, monoclonal and polyclonal antibodies, modified proteins and fragments thereof.

As used herein, the term “modulates” refers the effect an agent, including a gene product, has on another agent, (e.g., STIM1 and/or ORAI). In one embodiment, an agent that modulates another agent upregulates or increases the activity of the second agent. In one embodiment, an agent that modulates another agent downregulates or decreases the activity of the second agent.

As used herein, the “contacting” is meant to be performed under conditions appropriate for assay performance. In one embodiment, contacting is under conditions appropriate for binding of STIM to ORAI. In another embodiment, contacting is performed under conditions appropriate for activation of ORAI by STIM.

Aspects of the present invention relate to the determination that STIM1 is sufficient for activation of the ORAI1 calcium channel. Prior to the instant invention, experiments studying the activation of ORAI1 by STIM1 were performed in cells which possess a store-operated calcium entry pathway and therefore contained the full complement of proteins necessary. The possible participation of these cellular factors could not be excluded from the results of STIM1 activation of ORAI1. However, experiments detailed in the Examples section below were performed in a yeast system. Since yeast lack store operated calcium entry, as well as STIM1 and ORAI homologs, these experiments conclusively indicate that no other cellular factors are required for STIM1 regulation of ORAI1.

One aspect of the present invention relates to methods of identifying an agent that modulates STIM1 regulation of the ORAI calcium channel. Such methods can be performed in pure systems which do not contain significant amounts of other mammalian factors, or homologs thereof, that are known or suspected to modulate intracellular calcium (e.g., by modulation of the STIM1 modulation of ORAI1). Modulators of STIM1 activation of ORAI can be identified at the level of binding of STIM1 to ORAI or at the level of activation of ORAI1 by STIM1. One such system is the Pichia pastoris system, in which P. Pastoris expressing ORAI or a fragment or derivative thereof, is used to assay binding of STIM1 or a fragment or derivative thereof, in the presence and absence of a test agent, under conditions appropriate for STIM1-ORAI binding. Modulation of binding of STIM1 to ORAI in the presence of the test agent, compared to binding in the absence of the test agent, indicates that the agent modulates STIM1 binding of a functional ORAI channel. One such assay for STIM1-ORAI binding is a membrane flotation assay, detailed in the Example section below. Another such assay is a lanthanide binding assay, detailed in the Examples section below. The binding assay can further be performed in the presence of one or more additional proteins (e.g., mammalian) known or suspected to modulate ORAI activation.

Another such assay system is the S. cerevisiae secretory vesicle system which expresses the ORAI protein or a fragment or derivative thereof. This assay system has the advantage of detecting modulation of STIM1 activation of ORAI function, rather than just binding of STIM1 to ORAI. Such secretory vesicles can be contacted with STIM1, or a fragment or derivative thereof, under conditions appropriate for the activation of the ORAI channel by STIM1. The contact occurs in the presence of a test agent. Calcium release from the vesicles is then monitored and compared to the calcium release from appropriate control vesicles. A significant difference in the calcium release from the vesicles compared to the controls (lacking the test agent) indicates the test agent modulates the STIM1 regulation of the ORAI calcium channel.

Another aspect of the present invention relates to a fungal or yeast organism that comprises a recombinant, expressed ORAI protein or fragment or derivative thereof. In one embodiment, the organism has been genetically engineered to contain, in expressible form, a nucleic acid encoding the ORAI protein or fragment or derivative thereof. In one embodiment the organism is S. cerevisiae, in another embodiment, the organisms is P. pastoris. In one embodiment, the ORAI protein or fragment or derivative thereof is functional. However, expression of a non-functional ORAI channel, fragment or derivative thereof (e.g., ORAI1 (E106Q) mutant) will also be useful in many applications. In one embodiment, the ORAI protein or fragment or derivative thereof is sufficient to bind to STIM1. In one embodiment, the ORAI protein or fragment or derivative thereof is sufficient for calcium channel function. The determination of a functional ORAI protein channel can be made using techniques known in the art, such as Ca⁺ flux assays, described herein. Membranes, liposomes or secretory vesicles isolated from such organisms are also encompassed by the instant invention. The organisms, membranes, liposomes or secretory vesicles may further comprise one or more additional proteins or factors known or suspected to modulate STIM1 regulation of intracellular calcium. This additional protein or factor can be exogenously added, or it can be expressed by the organism from an exogenously added nucleic acid. In one embodiment, the ORAI protein or functional fragment or derivative thereof, constitutes from about 1%-5%, at least 5%, at least 10%, at least 20%, or greater of plasma membrane protein of the yeast organism, or of the isolated membrane, liposome, or vesicle thereof. Appropriate expression of a protein for incorporation into the yeast membrane is described in Monk et al., US 2009/0143308, the contents of which are incorporated herein in their entirety.

The yeast or fungal organism, or microsomal membranes or secretory vesicles thereof, may further comprise one or more calcium detection agents. In one embodiment, the organism functionally expresses (e.g., by genetic engineering to) such a detection agent, for example, the S. cerevisiae or secretory vesicles may further comprise a recombinant functionally expressed calcium sensor, such as a chameleon calcium sensor (e.g., D3 cpV or D4 cpV).

Another aspect of the present invention relates to a system for detection of calcium release or STIM1-ORAI binding. The system contains an expressed recombinant ORAI or fragment or derivative thereof (e.g., a functional channel) The system may also contain a STIM1, or a functional fragment or derivative thereof (e.g., functional). In one embodiment, the system contains no significant amounts of additional mammalian proteins or factors. Such a system can be derived from yeast or other fungi, or another organisms such as a non-mammalian organism (e.g., single cell, multi-cellular). Preferably, it is derived from an organism that does not have store operated Ca⁺⁺ entry, and/or has no significant reservoir of calcium in the endoplasmic reticulum, and/or does not posses orthologues of the ER calcium-ATPase or IP3 receptor, and/or has no STIM1 or ORAI homologs or orthologues. The system may further comprise a calcium detection agent. The system may further comprise one or more added mammalian proteins or factors known or suspected to contribute to the regulation of STIM1 activation of ORAI or known or suspected to participate in calcium channel regulation.

In one embodiment, the system is an ORAI protein, fragment or derivative thereof, reconstituted into liposomes. In one embodiment, the ORAI protein is purified or partially purified. In one embodiment, the ORAI protein, fragment or derivative, is obtained from an organism that does not have store operated Ca⁺⁺ entry, and/or has no significant reservoir of calcium in the endoplasmic reticulum, and/or does not posses orthologues of the ER calcium-ATPase or IP3 receptor, and/or has no STIM1 or ORAI homologs or orthologues (e.g., a fungus or yeast genetically engineered to expressed the ORAI protein). Such liposomes can be used in the assays described herein (e.g, in place of the secretory vesicles or microsomal membranes, or membranes isolated from yeast or other fungal species expressing the ORAI protein). Procedures for reconstitution of channel proteins into liposomes are provided in Goldberg AFX and Miller C (1991) J Membrane Biol 124, 199-206; Maduke M, Pheasant D J and Miller C (1999) J Gen Physiol 114, 713-722; Heginbotham L, Kolmakova-Partensky L and Miller C (1998) J Gen Physiol 111, 741-749, the contents of which are incorporated herein by reference.

Another example of such a system is a recombinant ORAI protein, fragment or derivative thereof, expressed (e.g., functionally) in S. cerevisiae, or the secretory vesicles thereof, (as described herein) and optionally STIM 1, or a fragment or derivative thereof (e.g., functional). Another example of such a system is a recombinant ORAI protein, fragment or derivative thereof, expressed (e.g., functionally) in P. pastoris, or the microsomal membrane thereof, (as described herein) and optionally STIM 1, or a fragment or derivative thereof (e.g., functional).

Vesicles, microsomal membranes, membrane fractions, or liposomes described herein, which contain the ORAI protein, fragment or derivative thereof, can be further processed into planar lipid bilayers. These lipid bilayers can be used for various assays of STIM-ORAI function (e.g., electrophysiological assays).

Vesicles, membranes, membrane fractions or liposomes described herein, which contain the ORAI protein, fragment or derivative thereof, can be used in binding assays, for binding to STIM1 or a fragment or derivative thereof. Such binding assays can be performed in the presence and absence of a test agent, to determine if the test agent modulates (increases or decreases) STIM1 binding to the ORAI protein. In such binding assays, STIM can be labeled for easy detection and/or isolation (e.g., radiolabelled, fluorescently or bioluminescently, etc.). Separation of the membranes with bound STIM1 from unbound STIM can be performed by an number of methods known in the art (e.g., filtration, centrifugation through oil).

Methods described herein relating to ORAI calcium channels expressed in the context of secretory vesicles or liposomes, which are then contacted with STIM1 (e.g., STIM1) or a functional fragment or derivative thereof, may alternatively be performed by contacting ORAI with STIM (e.g., STIM1) or a functional fragment thereof expressed in the context of a secretory vesicle or liposome, to thereby generate a functional ORAI channel, to produce comparable results. Similarly, systems expressing recombinant STIM (e.g., STIM1) or a functional fragment thereof, in a vesicle or membrane or liposome, and further including a recombinant ORAI, are also encompassed by the present invention.

### Store Operated Calcium Entry (SOCE)

SOCE is one of the main mechanisms to increase intracellular cytoplasmic free Ca²⁺ concentrations ([Ca²⁺]i) in electrically non-excitable cells. Ca²⁺ elevations are a crucial signal transduction mechanism in virtually every cell. The tight control of intracellular Ca²⁺, and its utility as a second messenger, is emphasized by the fact that [Ca²⁺]i levels are typically 70-100 nM while extracellular Ca²⁺ levels ([Ca²⁺]ex) are 10⁴-fold higher, ˜1-2 mM. The immediate source of Ca²⁺ for cell signaling can be either intracellular or extracellular. Intracellular Ca²⁺ is released from ER stores by inositol 1,4,5-triphosphate (IP3), or other signals, while extracellular Ca²⁺ enters the cell through voltage-gated, ligand-gated, store-operated or second messenger-gated Ca²⁺ channels in the plasma membrane. In electrically non-excitable cells such as lymphocytes, the major mechanism for Ca²⁺ entry is store-operated Ca²⁺ entry, a process controlled by the filling state of intracellular Ca²⁺ stores. Depletion of intracellular Ca²⁺ stores triggers activation of membrane Ca²⁺ channels with specific electrophysiological characteristics, which are referred to as calcium release-activated Ca²⁺ (CRAC) channels (Parekh and Putney, Jr. 2005, Physiol Rev 85:757).

Ca²⁺ release activated Ca²⁺ (CRAC) channels. The electrophysiological characteristics of CRAC channels have been studied intensively. One definition of CRAC channels holds that depletion of intracellular Ca²⁺ stores is both necessary and sufficient for channel activation without direct need for increases in [Ca²⁺]i, inositol phosphates IP3 or IP4, cGMP or cAMP (Parekh and Penner. 1997, Physiol Rev. 77:901). Biophysically, CRAC current is defined, amongst other criteria, by its activation as a result of ER Ca²⁺ store depletion, its high selectivity for Ca²⁺ over monovalent (Cs⁺, Na⁺) cations, a very low single channel conductance, a characteristic I-V relationship with pronounced inward rectification and its susceptibility to pharmacological blockade for instance by La³⁺ and 2-APB (100 μM), respectively (Parekh and Putney, Jr. 2005, Physiol Rev 85:757; Lewis, 2001, Annu Rev Immunol 19:497).

### Downstream Calcium Entry-Mediated Events

In addition to intracellular changes in calcium stores, store-operated calcium entry affects a multitude of events that are consequent to or in addition to the store-operated changes. For example Ca²⁺ influx results in the activation of a large number of calmodulin-dependent enzymes including the serine phosphatase calcineurin. Activation of calcineurin by an increase in intracellular calcium results in acute secretory processes such as mast cell degranulation. Activated mast cells release preformed granules containing histamine, heparin, TNFα and enzymes such as β-hexosaminidase. Some cellular events, such as B and T cell proliferation, require sustained calcineurin signaling, which requires a sustained increase in intracellular calcium. A number of transcription factors are regulated by calcineurin, including NFAT (nuclear factor of activated T cells), MEF2 and NF κB. NFAT transcription factors play important roles in many cell types, including immune cells. In immune cells NFAT mediates transcription of a large number of molecules, including cytokines, chemokines and cell surface receptors. Transcriptional elements for NFAT have been found within the promoters of cytokines such as IL-2, IL-3, IL-4, IL-5, IL-8, IL-13, as well as tumor necrosis factor alpha (TNFα), granulocyte colony-stimulating factor (G-CSF), and gamma-interferon (γ-IFN).

The activity of NFAT proteins is regulated by their phosphorylation level, which in turn is regulated by both calcineurin and NFAT kinases. Activation of calcineurin by an increase in intracellular calcium levels results in dephosphorylation of NFAT and entry into the nucleus. Rephosphorylation of NFAT masks the nuclear localization sequence of NFAT and prevents its entry into the nucleus. Because of its strong dependence on calcineurin-mediated dephosphorylation for localization and activity, NFAT is a sensitive indicator of intracellular calcium levels.

### Calcium Signaling Associated Diseases

The methods of the present invention can also be utilized to identify agents useful in treatment of, conditions and diseases associated with disregulation/disfunction of Calcium signaling. Such diseases include, without limitation, immune system diseases involving hyperactivity or inappropriate activity of the immune system, e.g., acute immune diseases, chronic immune diseases and autoimmune diseases Examples of such diseases include rheumatoid arthritis, inflammatory bowel disease, allogeneic or xenogeneic transplantation rejection (organ, bone marrow, stem cells, other cells and tissues), graft-versus-host disease, aplastic anemia, psoriasis, lupus erythematosus, inflammatory disease, MS, type I diabetes, asthma, pulmonary fibrosis, scleroderma, dermatomyositis, Sjogren's syndrome, postpericardiotomy syndrome, Kawasaki disease, Hashimoto's thyroiditis, Graves' disease, myasthenia gravis, pemphigus vulgaris, autoimmune hemolytic anemia, idiopathic thrombopenia, chronic glomerulonephritis, Goodpasture's syndrome, Wegner's granulomatosis, multiple sclerosis, cystic fibrosis, chronic relapsing hepatitis, primary biliary cirrhosis, uveitis, allergic rhinitis, allergic conjunctivitis, atopic dermatitis, Crohn's disease, ulcerative colitis, colitis/inflammatory bowel syndrome, Guilllain-Barre syndrome, chronic inflammatory demyelinating polyradiculoneuropathy, eczema, and autoimmune thyroiditis. Transplant graft rejections can result from tissue or organ transplants. Graft-versus-host disease can result from bone marrow or stem cell transplantation. Immune system diseases involving hypoactivity of the immune system include, e.g., immunodeficiency diseases including acquired immunodeficiencies, such as HIV disease, and common variable immunodeficiency (CVID).

The methods of the present invention can also be utilized to identify agents useful in treatment of conditions and diseases that are not immune mediated, but which nevertheless involve aberrant calcium signaling, such as aberrant Ca²⁺⁻ calcineurin-mediated activation of NFAT, e.g. a protein-protein interaction between calcineurin and NFAT. Examples include myocardial hypertrophy, dilated cardiomyopathy, excessive or pathological bone resorption, excessive adipocyte differentiation, obesity, and reactivation of latent human herpesvirus-8 or other viruses. Further, the methods of the present invention can be utilized to treat, or identify agents useful in the treatment of, conditions that involve a dysfunction of cellular Ca²⁺ signaling, such as those attributable to altered function of STIM1 or ORAI, wherein, the dysfunction of Ca²⁺ signaling causes a disease or disorder at least in part through its effects on other Ca²⁺ dependent pathways in addition to the STIM1-ORAI pathway, or wherein the dysfunction of Ca²⁺ signaling acts largely through such other pathways and the changes downstream of regulation of ORAI channel are ancillary.

### Severe Combined Immunodeficiency

One calcium signaling associated disease/disorder is Severe Combined Immunodeficiency (SCID). SCID is a group of congenital immune disorders caused by failed or impaired development and/or function of both T and B lymphocytes. A rare disease with an estimated prevalence of 1 per 100,000 population, SCID can be caused by mutations in more than 20 different genes. Mutations in the common γ chain (cγ) of the interleukin 2 (IL-2), IL-4, -7, -9 and -15 receptors leading to X-linked SCID account for 50% of all cases. Approximately 10% of all SCID cases are due to a variety of rare mutations in genes important for T and B cell development or function, especially signal transduction (CD38 and γ, ZAP-70, p561ck, CD45, JAK3, IL-7Rα chain). Due to the low incidence of these mutations and small family sizes, classical positional cloning is usually not possible for most of these SCID diseases and mutations were often found in known signal transducing genes by functional analysis of T cells followed by sequencing of candidate genes. Scientifically, SCID disease has been of extraordinary value for the elucidation of T cell and B cell function, highlighting the consequences of gene dysfunction in the immune system. SCID patients have been found to possess a missense mutation in Exon 1 or ORAI1. Specifically, the mutation at position 271 of the coding sequence of Orai1 (position 444 of NM_{—}032790), a C>A transition, leads to substitution of tryptophan for a highly-conserved arginine residue at position 91 (R91W) of the protein. This point mutation is responsible for the genetic defects in store-operated calcium entry and I_{CRAC} function in some patients with a rare form of SCID.

The invention relates to screening methods (also referred to herein as “assays”) for identifying modulators, i.e., candidate compounds or agents (e.g., proteins, peptides, peptidomimetics, peptoids, oligonucleotides (such as siRNA or anti-sense RNA), small non-nucleic acid organic molecules, small inorganic molecules, or other drugs) of STIM1 regulation of the ORAI calcium channel. Such interacting proteins can include Ca²⁺ and other subunits of calcium channels, proteins that interact with one or more Orai proteins, e.g., additional CRAC channel subunits or CRAC channel modulatory proteins. The modulator compounds can be novel, compounds not previously identified as having any type of activity as a calcium channel modulator, or a compound previously known to modulate calcium channels, but that is used at a concentration not previously known to be effective for modulating calcium influx.

Compounds that modulate the activity of ORAI are useful in the treatment of disorders involving cells that express the ORAI. Particularly relevant disorders are those involving hyperactivity or inappropriate activity of the immune system or hypoactivity of the immune system, as further described herein.

### Test Compounds

The test compounds or agents for use in the methods of the present invention can be obtained using any of the numerous approaches in combinatorial library methods known in the art, including: biological libraries; peptoid libraries (libraries of molecules having the functionalities of peptides, but with a novel, non-peptide backbone, which are resistant to enzymatic degradation but that nevertheless remain bioactive; see, e.g., Zuckermann, et al., 1994 J. Med. Chem. 37: 2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam (1997) Anticancer Drug Des. 12:145).

The compounds that can be screened by the methods described herein include, but are not limited to, any small molecule compound libraries derived from natural and/or synthetic sources, small non-nucleic acid organic molecules, small inorganic molecules, peptides, peptoids, peptidomimetics, oligonucleotides (e.g., siRNA, antisense RNA, aptamers such as those identified using SELEX), and oligonucleotides containing synthetic components.

In addition to screening test agents to initially identify activity of a molecule, the methods of the present invention can further help classify agents known to modify store operated calcium entry, to further determine their mechanism of action. Such agents can be naturally occurring molecules in the cell that participate in calcium channel regulation, other molecules that are identified as interacting with STIM1-ORAI (e.g., in binding studies), or any naturally occurring molecule suspected to play a role in calcium mediated signaling. Such agents can also be agents not present naturally in the cell, but known to affect store mediated calcium entry, e,g, identified in other assays or screens. In such a method, one or more such agents are contacted to the assay systems described herein, under conditions appropriate for STIM1-ORAI binding, and/or activity. Agents that are identified as directly affecting the STIM1-ORAI interaction can further be used as bait in screens to identify modulators of the ORAI calcium channel. They can also further be used to identify other such agents that participate in STIM1-ORAI regulation (e.g, naturally occurring in the cell).

The test compounds can be administered, for example, by diluting the compounds into the solution or medium wherein the assay system is maintained.

A variety of other reagents may also be included in the mixture. These include reagents such as salts, buffers, neutral proteins, e.g. albumin, detergents, etc. which may be used to facilitate optimal protein-protein and/or protein-nucleic acid binding and/or reduce non-specific or background interactions, etc. Also, reagents that otherwise improve the efficiency of the assay, such as protease inhibitors, nuclease inhibitors, antimicrobial agents, etc. may be used.

### Assays for Modulation of Calcium Levels

In monitoring the effect of a test agent on intracellular calcium in any of the screening/identification methods provided herein, a direct or indirect evaluation or measurement of cellular (including cytosolic and intracellular organelle or compartment) calcium and/or movement of ions into, within or out of a cell, organelle, or portions thereof (e.g., a membrane) can be conducted. A variety of methods are described herein and/or known in the art for evaluating calcium levels and ion movements or flux. The particular method used and the conditions employed can depend on whether a particular aspect of intracellular calcium is being monitored. For example, as described herein, reagents and conditions are known, and can be used, for specifically evaluating store-operated calcium entry, resting cytosolic calcium levels and calcium levels and uptake by or release from intracellular organelles or secretory vesicles (e.g., isolated) via STIM1-ORAI interactions. The effect of test agent on calcium movement via the ORAI channel can be monitored using, for example, a cell, an intracellular organelle or storage compartment, a membrane (including, e.g., a detached membrane patch or a lipid bilayer) or a cell-free assay system.

Generally, monitoring the effect of a test agent on intracellular calcium involves contacting a test agent with or exposing a test agent to (1) a protein involved in modulating ORAI calcium channel activity (e.g., STIM1) and/or (2) a cell, or portion(s) thereof (e.g., a membrane or intracellular structure or organelle, isolated secretory vesicle) that contains a protein involved in modulating ORAI calcium channel activity (e.g., STIM1). A cell, membrane, or intracellular structure, organelle or isolated secretory vesicle, can be one that exhibits one or more aspects of intracellular Ca²⁺ modulation, such as, for example, ORAI mediated calcium transport. Before, during and/or after the contacting of test agent, a direct or indirect assessment of intracellular calcium can be made. An indirect assessment can be, for example, evaluation or measurement of current through an ion transport protein (e.g., a store-operated calcium channel or a Ca²⁺-regulated ion channel), or transcription of a reporter protein operably linked to a calcium-sensitive promoter. A direct assessment can be, for example, evaluation or measurement of intracellular (including cytosolic and intracellular organelle) calcium.

An assessment of intracellular calcium conducted to monitor the effect of test compound on intracellular calcium can be made under a variety of conditions. Conditions can be selected to evaluate the effect of test compound on a specific aspect of intracellular calcium. For example, as described herein, reagents and conditions are known, and can be used, for specifically evaluating store-operated calcium entry, resting cytosolic calcium levels and calcium levels of and calcium uptake by or release from intracellular organelles or other membrane derived/containing systems described herein. For example, as described herein, calcium levels and/or calcium release from the endoplasmic reticulum, vesicles, microsomal membranes, or liposomes described herein, can directly be assessed using mag-fura 2, endoplasmic reticulum-targeted aequorin or cameleons. One method for indirect assessment of calcium levels or release is monitoring intracellular cytoplasmic calcium levels (for example using fluorescence-based methods) after exposing a cell to an agent that effects calcium release (actively, e.g., IP₃, or passively, e.g., thapsigargin) from the organelle in the absence of extracellular calcium. Assessment of the effect of the test agent/compound on concentrations of cations or divalent cations within the cell, or of ion influx into the cell, can also be used to identify a test agent as an agent that modulates intracellular calcium.

Resting cytosolic calcium levels, intracellular organelle calcium levels and cation movement may be assessed using any of the methods described herein or known in the art (see, e.g., descriptions herein of calcium-sensitive indicator-based measurements, such as fluo-3, mag-fura 2 and ER-targeted aequorin, labeled calcium (such as ⁴⁵Ca²⁺)-based measurements, and electrophysiological measurements). Particular aspects of ion flux that may be assessed include, but are not limited to, a reduction (including elimination) or increase in the amount of ion flux, altered biophysical properties of the ion current, and altered sensitivities of the flux to activators or inhibitors of calcium flux processes, such as, for example, store-operated calcium entry. Reagents and conditions for use in specifically evaluating receptor-mediated calcium movement and second messenger-operated calcium movement are also available, some of which are described herein. Ion flux assays are preferably carried out by measuring Ca²⁺ flux, but can also be carried out under modified conditions by measuring fluxes or currents carried by alternative ions such as Na⁺, Li⁺, Sr²⁺, or Ba²⁺.

For example, a fluorescent calcium indicator (e.g., FLUO-4). calcium movement across membranes is detected depending on the specific indicator used as, e.g. an increase in fluorescence or bioluminescence, a decrease in fluorescence or bioluminescence, or a change in the ratio of fluorescence or bioluminescence intensities elicited by excitation using light of two different wavelengths. in response to conditions under which store-operated calcium entry occurs. The methods for eliciting the fluorescence signal for a specific calcium indicator and for interpreting its relation to a change in free calcium concentration are well known in the art. The conditions include addition of a store-depletion agent, e.g., thapsigargin (which inhibits the ER calcium pump and allows discharge of calcium stores through leakage) to the media of cell that has been incubated in Ca²⁺-free buffer, incubation with thapsigargin for about 5-15 minutes, addition of test compound (or vehicle control) to the media and incubation of the cell with test agent for about 5-15 minutes, followed by addition of external calcium to the media to a final concentration of about 1.8 mM. By adding thapsigargin to the cell in the absence of external calcium, it is possible to delineate the transient increase in intracellular calcium levels due to calcium release from calcium stores and the more sustained increase in intracellular calcium levels due to calcium influx into the cell from the external medium (i.e., store-operated calcium entry through the plasma membrane that is detected when calcium is added to the medium). Because the luminescence- or fluorescence-based assay allows for essentially continuous monitoring of calcium movement during the entire period of a given event, (e.g., from prior to addition of thapsigargin until well after addition of calcium to the medium), not only can “peak” or maximal calcium levels resulting from store-operated calcium entry be assessed in the presence and absence of test agent, a number of other parameters of the calcium entry process may also be evaluated, as described herein. For example, the kinetics of store-operated calcium entry can be assessed by evaluation of the time required to reach peak calcium levels, the up slope and rate constant associated with the increase in calcium levels, and the decay slope and rate constant associated with the decrease in calcium levels as store-operated calcium entry discontinues. Any of these parameters can be evaluated and compared in the presence and absence of test agent to determine whether the agent has an effect on store-operated calcium entry, and thus on intracellular calcium. In other embodiments, store-operated calcium entry can be evaluated by, for example, assessing a current across a membrane or into a cell that is characteristic of a store-operated calcium entry current (e.g., responsiveness to reduction in calcium levels of intracellular stores) or assessing transcription of a reporter construct that includes a calcium-sensitive promoter element. In particular embodiments, a test agent is identified as one that produces a statistically significant difference. E.g., at least a 30% difference in any aspect or parameter of store-operated calcium entry relative to control (e.g., absence of compound, i.e., vehicle only).

Generally, a test agent is identified as an agent, or candidate agent, that modulates intracellular calcium if there is a detectable effect of the agent on intracellular calcium levels and/or ion movement or flux, such as a detectable difference in levels or flux in the presence of the test agent. In particular embodiments, the effect or differences can be substantial or statistically significant. A test agent is identified as an agent that modulates binding if there is a statistically significant difference in detected binding in a given binding assay, when compared to an appropriate control.

Direct testing of the effect of a test compound on the activity of an ORAI channel can be accomplished using, e.g., patch clamping to measure I_{CRAC}. This method can be used in screening assays as a second step after testing for general effects on calcium flux or as a second step after identifying a test compound as affecting STIM1 regulation of ORAI. Alternatively, direct testing can be used as a first step in a multiple step assay or in single step assays.

Many such monitoring agents are known in the art. The term “monitoring agent” is also meant to include any apparatus used for such monitoring.

In particular embodiments of the systems, the ORAI protein or fragment or derivative thereof, is contained in isolated membranes, or vesicles obtained from a cell (e.g., yeast or other fungal cell) that expresses the protein. The cells recombinantly express such proteins as described above e.g. a recombinant cell overexpressing at least one ORAI protein or fragment or derivative thereof.

### Recombinant Cells

Aspects of the invention further relate to recombinant cells used in the assays described in the methods discussed herein. In one aspect, the invention also encompasses any recombinant cells described herein. In one embodiment, the recombinant cell comprises at least one exogenous (heterologous or homologous) ORAI protein or fragment or derivative thereof. The recombinant cell may also further comprise at least one exogenous (heterologous or homologous) nucleic acid encoding the ORAI protein or fragment or derivative thereof. The recombinant cell may be of eukaryotic or prokaryotic origin. Without limitation, they can be or mammalian, yeast, plant, or insect origin. The recombinant cell may over express the ORAI protein or fragment or derivative thereof. This overexpression may result from expression of an exogenous (heterologous or homologous) ORAI protein (e.g. from an exogenous nucleic acid) of when the cell contains endogenous ORAI, may result from over expression of native/endogenous ORAI.

### Stromal Interaction Molecule 1 (STIM1)

STIM1 plays an important role in store operated Ca²⁺ entry and CRAC channel function. Three independent RNAi screens by Roos et al. (2005, J Cell Biol 169:435), Liou et al. (2005, Curr Biol 15:1235) have found that suppression of STIM expression by RNAi impairs Ca²⁺ influx in Drosophila melanogaster S2 cells as well as mammalian cells. STIM1 is a type I transmembrane protein which was initially characterized as a stromal protein promoting the expansion of pre-B cells and as a putative tumor suppressor (Oritani, et al. 1996. J Cell Biol 134:771; Sabbioni, et al. 1997. Cancer Res 57:4493). The human gene for STIM1 is located on chromosome 11p15.5 which is believed to contain genes associated with a number of pediatric malignancies, including Wilms tumor (Parker et al. 1996, Genomics 37:253). STIM1 contains a Ca²⁺ binding EF hand motif and a sterile α-motif (SAM) domain in its ER/extracellular region, a single membrane-spanning domain, and two predicted cytoplasmic coiled-coil regions (Manji et al. 2000, Biochim Biophys Acta 1481:147). Domain structure and genomic organization are conserved in a related gene called STIM2, which differs from STIM1 mainly in its C-terminus (Williams et al. 2002, Biochim Biophys Acta 1596:131). STIM1 is able to homodimerize or heterodimerize with STIM2 (Williams et al. 2002 supra). Expressed in the ER, its C-terminal region is located in the cytoplasm whereas the N-terminus resides in the lumen of the ER, as judged by glycosylation and phosphorylation studies (Maji et al. 2000 supra; Williams et al. 002 supra). A minor fraction of STIM1 is located in the plasma membrane. Although RNAi mediated suppression of STIM1 expression interferes with SOCE and CRAC channel function, STIM1 is not a Ca²⁺ channel itself. Rather STIM1 senses Ca²⁺ levels in the ER via its EF hand (Putney, Jr. 2005. J Cell Biol 169:381; Marchant, 2005, Curr Biol 15:R493). Consistent with the conformational coupling model of store-operated Ca²⁺ influx, STIM1 acts as a key adapter protein, which physically bridges the space between ER and plasma membrane, and thus directly connects sensing of depleted Ca²⁺ stores to store-operated Ca²⁺ channels in the plasma membrane (Putney, Jr. 2005. supra; Putney, Jr. 1986, Cell Calcium 7:1).

STIM1 senses a reduction of ER luminal Ca²⁺ concentration through dissociation of Ca²⁺ from a luminal EF-hand, leading to oligomerization of STIM, and then a local redistribution within the ER by which STIM becomes enriched at sites of ER-plasma membrane apposition, termed puncta (Liou, J., et al., Curr Biol 15, 1235-1241 (2005); Zhang, S. L., et al., Nature 437, 902-905 (2005); Spassova, M. A., et al., Proc Natl Acad Sci USA 103, 4040-4045 (2006); Mercer, J. C., et al., J Biol Chem 281, 24979-24990 (2006); Wu, M. M., et al., J Cell Biol 174, 803-813 (2006); Baba, Y., et al., Proc Natl Acad Sci USA 103, 16704-16709 (2006); Liou, J., et al., Proc Natl Acad Sci USA 104, 930 1-9306 (2007); Ong, H. L., et al., J Biol Chem 282, 121 76-12185 (2007)). Subsequently, STIM1 recruits ORAI1 to ER-plasma membrane contacts, where Ca²⁺ enters the cell through opened ORAI channels (Xu, P., et al., Biochem Biophys Res Comm 350, 969-976 (2006); Luik, R. M., et al., J Cell Biol 174, 815-825 (2006); Luik, R. M., et al., Nature 454, 538-542 (2008); Muik, M., et al., et al., J Biol Chem 283, 8014-8022 (2008); Navarro-Borelly, L., et al., J Physiol 586, 5383-5401 (2008)). Structural and biochemical studies with recombinant ER-luminal portions of STIM 1 and STIM2 have illuminated the molecular mechanism by which STIM proteins sense Ca²⁺ changes in the ER lumen (Stathopulos, P. B., et al., J Biol Chem 281, 35855-35862 (2006); Stathopulos, P. B., et al., Cell 135, 110-122 (2008)).

The methods and compositions described herein may contain STIM1, STIM2, or a fragment or derivative thereof. In one embodiment, the fragment or derivative thereof binds to ORAI (e.g., ORAI1, ORAI2, or ORAI3). In one embodiment, the fragment or derivative used retains the activity of activating ORAI1, as described herein. It is to be understood that the methods and compositions described herein as performed or containing STIM1 or a fragment or derivative thereof, may alternatively be performed or contain STIM2 or a fragment or derivative thereof. The STIM1 and STIM2, or fragments or derivatives thereof, may be from any organism which expresses a homolog. This includes, without limitation, from mammalian (e.g., human, rat, mouse), vertebrate, insects (e.g., drosophila).

### Orai Proteins

As used herein, the term ORAI encompasses the ORAI homologs, (e.g., ORAI1, ORAI2 and ORAI3). The methods and compositions described herein may contain ORAI protein or a fragment or derivative thereof. In one embodiment, the fragment or derivative thereof binds to STIM1. In one embodiment, the fragment or derivative used retains the calcium channel activity, as described herein.

There are three known ORAI genes, which encode the respective ORAI proteins. ORAI1 nucleic acid sequence corresponds to GenBank accession number NM_{—}032790, ORAI2 nucleic acid sequence corresponds to GenBank accession number BC069270 and ORAI3 nucleic acid sequence corresponds to GenBank accession number NM_{—}152288. As used herein, ORAI used in the context of a protein, refers to the expressed protein product of any one of the ORAI genes, e.g., ORAI1, ORAI2, ORAI3. Any method or compositions described herein as using or containing an ORAI protein, may use or contain any one or more of ORAI1, ORAI2, ORAI3. The ORAI protein, or fragments or derivatives thereof, used in the methods and compositions described herein, may further be from any organism which expresses their relevant orthologue/homolog. This includes, without limitation, from mammals (e.g., human, rat, mouse), vertebrates, and insects (e.g., drosophila). The ORAI fragments may be functional fragments, in that they retain function as calcium channel pores, or they may be non-functional as calcium channel pores, but retain some useful property (e.g., binding to STIM1).

Orai1 (also known as CRACM1) is a widely expressed, 33 kDa plasma membrane protein with 4 transmembrane domains and a lack of significant sequence homology to other ion channels (Vig, M. et al. Science 312, 1220-1223 (2006); Zhang, S. L. et al. Proc. Natl. Acad. Sci. USA 103, 9357-9362 (2006)). Studies of T cells from human patients with a severe combined immunodeficiency (SCID) syndrome, in which T cell receptor engagement or store depletion failed to activate Ca²⁺ entry, was shown to be due to a single point mutation in Orai1 (Feske, S. et al. Nature 441, 179-185 (2006)). Other mammalian Orai homologues exist, e.g. Orai2 and Orai3. Orai2 and Orai3 can exhibit SOC channel activity when overexpressed with STIM1 in HEK cells (Mercer, J. C. et al. J. Biol. Chem. 281, 24979-24990 (2006)).

Evidence that Orai1 contributes to the CRAC channel pore was obtained by Orai1 mutagenesis studies. Selectivity of the CRAC channel for Ca²⁺ ions was shown by mutations at either Glu 106 or Glu 190, which weaken the ability of Ca²⁺ binding in order block permeation of monovalent cations (similar to mechanisms described for voltage-gated Ca²⁺ channels) (Yeromin, A. V. et al. Nature 443, 226-229 (2006); Vig, M. et al. Curr. Biol. 16, 2073-2079 (2006); Prakriya, M. et al. Nature 443, 230-233 (2006)).

Neutralizing the charge on a pair of aspartates in the I-II loop (Asp 110 and Asp 112) reduces block by Gd³⁺ and block of outward current by extracellular Ca²⁺, indicating that these negatively charged sites may promote accumulation of polyvalent cations near the mouth of the pore.

Currents observed through overexpression of Orai1 closely resemble I_{CRAC}, and the fact that Orai1 can form multimers (Yeromin, A. V. et al. Nature 443, 226-229 (2006); Vig, M. et al. Curr. Biol. 16, 2073-2079 (2006); Prakriya, M. et al. Nature 443, 230-233 (2006)), indicates that the native CRAC channel is either a multimer of Orai1 alone or in combination with the closely related subunits Orai2 and/or Orai3.

### Fusion Proteins and Epitope Tags

Proteins of the present invention can be modified to form a fusion protein or to contain an identifying epitope tag. In one embodiment, the fusion polypeptides comprise a STIM protein or an ORAI protein and a second heterologous polypeptide to increase the stability of the fusion polypeptide, or to modulate its biological activity or localization, or to facilitate purification of the fusion polypeptide. Exemplary heterologous polypeptides that can be used to generate fusion polypeptides include, but are not limited to, polyhistidine, Glu-Glu, glutathione S transferase (GST), thioredoxin, polypeptide A, polypeptide G, and an immunoglobulin heavy chain constant region (Fc), maltose binding polypeptide (MBP), which are particularly useful for isolation of the fusion polypeptides by affinity chromatography. For the purpose of affinity purification, relevant matrices for affinity chromatography, such as glutathione-, amylase-, and nickel- or cobalt-conjugated resins are used. Another fusion domain well known in the art is green fluorescent polypeptide (GFP). Fusion domains also include “epitope tags,” which are usually short peptide sequences for which a specific antibody is available. Well known epitope tags for which specific monoclonal antibodies are readily available include FLAG, influenza virus haemagglutinin (HA), and c-myc tags.

### Yeast Expression Vectors

The nucleic acid (e.g., cDNA or genomic DNA) encoding STIM1 or ORAI1 may be inserted into a replicable vector for cloning (amplification of the DNA) or for expression in one or more specific organisms (e.g., a yeast or other fungal cell). Various vectors are publicly available. The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

Examples of suitable promoting sequences for use with yeast hosts include the promoters for 3-phosphoglycerate kinase [Hitzeman et al., J. Biol. Chem., 255:2073 (1980)] or other glycolytic enzymes [Hess et al., J. Adv. Enzyme Reg., 7:149 (1968); Holland, Biochemistry, 17:4900 (1978)], such as enolase, glyceraldehyde-3-phosphate dehydrogenase, hexokinase, pyruvate decarboxylase, phospho-fructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657.

Transcription of STIM1 or ORAI1 from vectors in mammalian host cells is controlled, for example, by promoters obtained from the genomes of viruses such as polyoma virus, fowlpox virus (UK 2,211,504 published 5 Jul. 1989), adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

Transcription of a DNA encoding STIM1 or ORAI1 by higher eukaryotes may be increased by inserting an enhancer sequence into the vector. Enhancers are cis-acting elements of DNA, usually about from 10 to 300 bp, that act on a promoter to increase its transcription. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. The enhancer may be spliced into the vector at a position 5′ or 3′ to the PRO533 coding sequence, but is preferably located at a site 5′ from the promoter.

### Vesicle Production and Isolation

Cells useful for expression of an ORAI protein and formation of vesicles to be used with the methods described herein can be derived from any known type of cells (e.g., mammalian, prokaryotic, eukaryotic, etc.) In one embodiment, they are from yeast or other fungal organisms, or other organisms which do not have store operated calcium entry, and/or contain STIM and/or ORAI orthologs (e.g., Saccharomyces cerevisiae, or Pichia pastoris).

In one embodiment, the yeast strain used with the methods described herein comprises S. cerevisiae, which provides a valuable system because its genome has been entirely sequenced and extensively annotated, thus its genetics are well understood. In one embodiment, the strain of yeast comprises sec6-4, a temperature sensitive mutant of Saccharomyces cerevisiae, which permits fusion of secretory vesicles with the plasma membrane at temperatures up to 30° C. When the yeast are grown at 37° C., a non-permissive temperature, the membrane fusion process is blocked and the membrane protein being expressed is retained in secretory vesicles.

ORAI1 can be expressed in a yeast system using any expression vector known to those of skill in the art for producing proteins in yeast.

Secretory vesicles comprising a membrane protein (e.g., ORAI1) are isolated from a culture of sec6-4 yeast as described herein and in the Example section. Briefly, cells are grown at 25° C. in uracil-deficient synthetic complete medium containing glucose, which was subsequently exchanged for galactose to induce transgene expression for a suitable time (e.g., 8 hours). The culture temperature is then adjusted to 37° C. to induce intracellular accumulation of secretory vesicles. Cells are collected by centrifugation and digested with e.g., Zymolase 20T enzyme. Spheroblasts are then harvested, coated with lectin and pelleted. The spheroblasts are then separated from unlysed cells, cell debris, mitochondria, and nuclei by centrifugation. A detailed exemplary method for vesicle isolation is described herein in the Examples Section.

### Flotation Assay

To determine the interaction of STIM1 and an ORAI protein or fragment or derivative thereof, a flotation assay is used herein. The assay is based on the premise that in the absence of an interaction STIM1 (a soluble protein) and ORAI1 (an integral membrane protein) are not present in the same fraction of a density gradient (e.g., sucrose gradient). However, an interaction between STIM1 and ORAI retains the STIM1 protein in the top portion of a density gradient, such that STIM1 and ORAI1 are present in the same fraction. Thus, essentially any density gradient that permits the separation of soluble and integral membrane proteins can be used with the methods described herein. In one embodiment, the density gradient comprises a sucrose gradient. This assay can also be used to determine the interaction of a test agent and ORAI by substituting the test agent for STIM1 in the assay described herein.

Exemplary methods useful for determining the interaction of STIM1 and ORAI1 as a marker for store-operated calcium release are described herein in the Examples section. Alternative membrane flotation assay methods can be found in Heyman, J A et al., J Cell Biol 127(5):1259-1273 (1994); Kim, J. et al., J Cell Biol 152(1):51-64 (2001); Huang, W.-P., et al., J. Biol. Chem. 275:5845-5851 (2000); and Noda, T., et al., J. Cell Biol. 148:465-480 (2000).

### Function Assays: Calcium Release

In monitoring the effect of a test agent on intracellular calcium in any of the screening/identification methods provided herein, a direct or indirect evaluation or measurement of cellular (including cytosolic and intracellular organelle or compartment) calcium and/or movement of ions into, within or out of a cell, organelle, or portions thereof (e.g., a membrane) can be conducted. A variety of methods are described herein and/or known in the art for evaluating calcium levels and ion movements or flux. The particular method used and the conditions employed can depend on whether a particular aspect of intracellular calcium is being monitored. For example, as described herein, reagents and conditions are known, and can be used, for specifically evaluating store-operated calcium entry, resting cytosolic calcium levels and calcium levels and uptake by or release from intracellular organelles. The effect of test agent on intracellular calcium can be monitored using, for example, a cell, an intracellular organelle or storage compartment, a membrane (including, e.g., a detached membrane patch or a lipid bilayer) or a cell-free assay system.

The assessment of intracellular calcium is made in such a way as to be able to determine an effect of an agent on intracellular calcium. Typically, this involves comparison of intracellular calcium in the presence of a test agent with a control for intracellular calcium. For example, one control is a comparison of intracellular calcium in the presence and absence of the test agent or in the presence of varying amounts of a test agent. Thus, one method for monitoring an effect on intracellular calcium involves comparing intracellular calcium before and after contacting a test agent with a test cell containing a protein that modulates intracellular calcium, or comparing intracellular calcium in a test cell that has been contacted with test agent and in a test cell that has not been contacted with test agent (i.e., a control cell). Generally, the control cell is substantially identical to, if not the same as, the control cell, except it is the cell in the absence of test agent. A difference in intracellular calcium of a test cell in the presence and absence of test agent indicates that the agent is one that modulates intracellular calcium.

In one embodiment, the cell does not express a protein or group of proteins involved in calcium store-released calcium, e.g., yeast or other fungi

### Detection of Ion Flux

An assessment of intracellular calcium conducted to monitor the effect of test compound on intracellular calcium can be made under a variety of conditions. Conditions can be selected to evaluate the effect of test compound on a specific aspect of intracellular calcium. For example, as described herein, reagents and conditions are known, and can be used, for specifically evaluating store-operated calcium entry, resting cytosolic calcium levels and calcium levels of and calcium uptake by or release from intracellular organelles. For example, as described herein, calcium levels and/or calcium release from the endoplasmic reticulum can directly be assessed using mag-fura 2, endoplasmic reticulum-targeted aequorin or cameleons. One method for indirect assessment of calcium levels or release is monitoring intracellular cytoplasmic calcium levels (for example using fluorescence-based methods) after exposing a cell to an agent that effects calcium release (actively, e.g., IP3, or passively, e.g., thapsigargin) from the organelle in the absence of extracellular calcium.

Resting cytosolic calcium levels, intracellular organelle calcium levels and cation movement may be assessed using any of the methods described herein or known in the art (see, e.g., descriptions herein of calcium-sensitive indicator-based measurements, such as fluo-3, mag-fura 2 and ER-targeted aequorin, labelled calcium (such as 45Ca2+)-based measurements, and electrophysiological measurements). Particular aspects of ion flux that may be assessed include, but are not limited to, a reduction (including elimination) or increase in the amount of ion flux, altered biophysical properties of the ion current, and altered sensitivities of the flux to activators or inhibitors of calcium flux processes, such as, for example, store-operated calcium entry.

Store-operated calcium entry into the cells is detected depending on the specific indicator used as, e.g. an increase in fluorescence, a decrease in fluorescence, or a change in the ratio of fluorescence intensities elicited by excitation using light of two different wavelengths. in response to conditions under which store-operated calcium entry occurs. The methods for eliciting the fluorescence signal for a specific calcium indicator and for interpreting its relation to a change in free calcium concentration are well known in the art. The conditions include addition of a store-depletion agent, e.g., thapsigargin (which inhibits the ER calcium pump and allows discharge of calcium stores through leakage) to the media of cell that has been incubated in Ca2+-free buffer, incubation with thapsigargin for about 5-15 minutes, addition of test compound (or vehicle control) to the media and incubation of the cell with test agent for about 5-15 minutes, followed by addition of external calcium to the media to a final concentration of about 1.8 mM. By adding thapsigargin to the cell in the absence of external calcium, it is possible to delineate the transient increase in intracellular calcium levels due to calcium release from calcium stores and the more sustained increase in intracellular calcium levels due to calcium influx into the cell from the external medium (i.e., store-operated calcium entry through the plasma membrane that is detected when calcium is added to the medium). Because the fluorescence-based assay allows for essentially continuous monitoring of intracellular calcium levels during the entire period from prior to addition of thapsigargin until well after addition of calcium to the medium, not only can “peak” or maximal calcium levels resulting from store-operated calcium entry be assessed in the presence and absence of test agent, a number of other parameters of the calcium entry process may also be evaluated, as described herein. For example, the kinetics of store-operated calcium entry can be assessed by evaluation of the time required to reach peak intracellular calcium levels, the up slope and rate constant associated with the increase in calcium levels, and the decay slope and rate constant associated with the decrease in calcium levels as store-operated calcium entry discontinues. Any of these parameters can be evaluated and compared in the presence and absence of test agent to determine whether the agent has an effect on store-operated calcium entry, and thus on intracellular calcium. In other embodiments, store-operated calcium entry can be evaluated by, for example, assessing a current across a membrane or into a cell that is characteristic of a store-operated calcium entry current (e.g., responsiveness to reduction in calcium levels of intracellular stores) or assessing transcription of a reporter construct that includes a calcium-sensitive promoter element. In particular embodiments, a test agent is identified as one that produces a statistically significant difference. E.g., at least a 30% difference in any aspect or parameter of store-operated calcium entry relative to control (e.g., absence of compound, i.e., vehicle only).

Generally, a test agent is identified as an agent, or candidate agent, that modulates intracellular calcium if there is a detectable effect of the agent on intracellular calcium levels and/or ion movement or flux, such as a detectable difference in levels or flux in the presence of the test agent. In particular embodiments, the effect or differences can be substantial or statistically significant.

Ion flux assays are preferably carried out by measuring Ca²⁺ flux, but can also be carried out under modified conditions by measuring fluxes or currents carried by alternative ions such as Na⁺, Li⁺, Sr²⁺, or Ba²⁺. In one embodiment, one or more of the cellular calcium assays described herein in the Examples section are used for testing a candidate agent.

### FRET-Based Calcium Sensor Systems

The use of genetically encoded fluorescent indicators for visualizing cellular calcium levels promises many advantages over fluorescent Ca-indicating dyes that have to be applied externally. Genetically encoded indicators are generated in situ inside cells after transfection, do not require cofactors, can in theory be specifically targeted to cell organelles and cellular microenvironments and do not leak out of cells during longer recording sessions. Furthermore, they are expressible within intact tissues of transgenic organisms and thus solve the problem of loading an indicator dye into tissue, while permitting labeling of specific subsets of cells of interest (for review see Zhang J., et al. “Creating new fluorescent probes for cell biology.” Nat. Rev. Mol. Biol. 3, 906-918 (2002)).

GFP-based calcium indicators can be used to monitor calcium flux in the methods described herein. In one embodiment, the GFP-based calcium indicator is a ratiometric indicator. In one embodiment, the indicator is a FRET pair of GFP variants with a linker that renders the fluorescence signal sensitive to calcium binding, such as a Chameleon. A chameleon is a pair of fluorescent proteins engineered for fluorescence resonance energy transfer (FRET) carrying the calcium binding protein calmodulin as well as a calmodulin target peptide sandwiched between the GFPs (see for example Miyawaki, A. et al. “Fluorescent indicators for Ca.sup.2+ based on green fluorescent proteins and calmodulin.” Nature 388, 882-887 (1997); Miyawaki, A. et al. “Dynamic and quantitative calcium measurements using improved cameleons.” Proc. Natl. Acad. Sci. USA 96, 2135-2140 (1999) and Truong et al. “FRET-based in vivo Ca²⁺ imaging by a new calmodulin-GFP fusion molecule.” Nat. Struct. Biol. 8, 1069-1073 (2001)). On another embodiment, the GFP-based calcium indicator is a non-ratiometric indicator with calmodulin directly inserted into a single fluorescent protein (see Baird, G. S. et al. “Circular permutation and receptor insertion within green fluorescent proteins.” Proc. Natl. Acad USA 96, 11241-11246 (1999); Nagai, T. et al. “Circularly permuted green fluorescent proteins engineered to sense Ca²⁺.” Proc. Natl. Acad. Sci. USA 98, 3197-3202 (2001); Nakai, J. et al. “A high signal-to-noise Ca²⁺ probe composed of a single green fluorescent protein.” Nat. Biotechnol. 19, 137-141 (2001); and Griesbeck, O. et al. “Reducing the environmental sensitivity of yellow fluorescent protein: mechanism and applications.” J. Biol. Chem. 276, 29188-29194 (2001)). Cameleon sensors can be obtained commercially from e.g., INVITROGENT™.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used to described the present invention, in connection with percentages means±1%.

In one respect, the present invention relates to the herein described compositions, methods, and respective component(s) thereof, as essential to the invention, yet open to the inclusion of unspecified elements, essential or not (“comprising). In some embodiments, other elements to be included in the description of the composition, method or respective component thereof are limited to those that do not materially affect the basic and novel characteristic(s) of the invention (“consisting essentially of”). This applies equally to steps within a described method as well as compositions and components therein. In other embodiments, the inventions, compositions, methods, and respective components thereof, described herein are intended to be exclusive of any element not deemed an essential element to the component, composition or method (“consisting of”).

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The present invention may be as defined in any one of the following numbered paragraphs.
1. A method of identifying an agent that modulates Ca²⁺ flux through the ORAI channel, comprising:
   a) providing S. cerevisiae secretory vesicles functionally expressing ORAI or a functional fragment or derivative thereof;
   b) contacting the secretory vesicles with STIM1, or a functional fragment or derivative thereof, and a test agent;
   c) monitoring calcium release from the vesicles;
      wherein a significant difference in the calcium release from the vesicles compared to a control which lacks the test agent, indicates the test agent modulates Ca²⁺ flux through the ORAI channel.
2. The method of paragraph 1, wherein the method is performed in the absence of other mammalian proteins.
3. The method or system of paragraphs 1 or 2, wherein the S. cerevisiae is a sec 6-4 strain.
4. The method of paragraphs 1-3, further comprising contacting the vesicles with another mammalian protein known to modulate STIM1 regulation of intracellular calcium.
5. The method of paragraphs 1-4, wherein monitoring step c) is with a calcium detection agent.
6. The method of paragraph 5, wherein the calcium detection agent is a fluorescent dye or FRET pairs of GFP variants sensitive to Ca⁺⁺ binding.
7. The method of paragraph 6, wherein the fluorescent dye or FRET pair is Fura-2, CFP and/or YFP.
8. The method of paragraphs 1-7, wherein the test agent is known to modulate intracellular calcium.
9. A method of identifying an agent that modulates ORAI regulation of intracellular calcium, comprising:
   a) providing yeast secretory vesicles or liposomes expressing a functional ORAI calcium channel;
   b) contacting the secretory vesicles or liposomes with a test agent;
   c) monitoring calcium release from the vesicles;
   wherein a significant difference in the calcium release from the vesicles compared to a control which lacks the test agent, indicates the test agent modulates ORAI regulation of intracellular calcium.
10. The method of paragraph 9, wherein contacting step b) further comprises contacting the secretory vesicles or liposomes with STIM1 or a functional fragment or derivative thereof.
11. The method of paragraph 1 or 10 wherein the STIM1 functional fragment is STIM1 (233-685), STIM1 (233-498), STIM1 (233-463), or STIM1 (233-600), and wherein the ORAI functional fragment is ORAI1 (65-301), ORAI1 (65-87), or full length ORAI1.
12. The method of paragraph 1 or 9, wherein the ORAI or functional fragment thereof comprises an epitope tag.
13. A system for detection of calcium release consisting essentially of:
   a) recombinant ORAI channel functionally expressed, and
   b) STIM1, functional fragment or derivative thereof.
14. A system comprising:
   a) a recombinant ORAI protein or fragment or derivative thereof, expressed in yeast or a vesicle or membrane isolated therefrom.
15. The system of paragraph 14, further comprising STIM1 protein, or a fragment or derivative thereof.
16. The system of paragraph 14 or 15, wherein the yeast is a S. cerevisiae or P. pastoris.
17. The system of paragraphs 13-16, wherein the ORAI protein or fragment or derivative thereof, and/or the STIM1 protein, or fragment or derivative thereof, is functional.
18. The system of paragraphs 13-17, further comprising a calcium detection agent.
19. The system of paragraphs 13-18, wherein the STIM1 protein, or fragment or derivative thereof, is STIM1 (233-685) STIM1 (233-498), STIM1 (233-463), or STIM1 (233-600), and wherein the ORAI protein or fragment or derivative thereof, is ORAI1 (65-301), ORAI1 (65-87), or full length ORAI1.
20. The system of paragraphs 13-19, which further comprises another mammalian protein or factor.
21. The system of paragraphs 13-19, which does not comprise another mammalian protein or factor.
22. A yeast organism, or microsomal membrane or vesicle thereof, that comprises a recombinant, expressed ORAI protein or fragment or derivative thereof.
23. The yeast organism of paragraph 22 that is genetically engineered to contain, in expressible form, a nucleic acid encoding the ORAI protein or fragment or derivative thereof.
24. The yeast organism, or microsomal membrane or vesicle thereof, of paragraph 22 that is S. cerevisiae, comprising a recombinant functionally expressed ORAI channel.
25. The yeast organism, or microsomal membrane or vesicle thereof, of paragraph 22 or 24 further comprising a recombinant functionally expressed calcium sensor.
26. The yeast organism, or microsomal membrane or vesicle thereof, of paragraph 24 or 25 wherein the S. cerevisiae is a sec6-4 strain.
27. The yeast organism, or microsomal membrane or vesicle thereof, of paragraphs 22-26, further comprising another mammalian protein known to modulate STIM1 regulation of intracellular calcium.
28. The yeast organism, or microsomal membrane or vesicle thereof, of paragraphs 22, 23, 27, that is Pichia pastoris.
29. A method of identifying an agent that modulates STIM1 binding of a functional ORAI channel, comprising:
   a) providing microsomal membranes prepared from P. Pastoris expressing ORAI or a functional fragment or derivative thereof; and
   b) performing a membrane flotation assay for binding of STIM1 or a fragment or derivative thereof, to the ORAI, in the presence and absence of a test agent; wherein modulation of binding of STIM1 to the ORAI in the presence of the test agent, compared to binding in the absence of the test agent, indicates that the agent modulates STIM1 binding of a functional ORAI channel.
30. A method of identifying an agent that modulates STIM1 binding of a functional ORAI channel, comprising:
   a) providing membranes with ORAI or a fragment or derivative thereof incorporated or reconstituted therein; and
   b) performing a binding assay for STIM1 or a fragment or derivative thereof, to
      the ORAI in the membrane, in the presence and absence of a test agent; wherein modulation of binding of STIM1 to the ORAI in the presence of the test agent, compared to binding in the absence of the test agent, indicates that the agent modulates STIM1 binding of a functional ORAI channel.
31. The method of paragraph 29 or 30, wherein ORAI is ORAI1.
32. The method of paragraphs 29-31, wherein the ORAI is a functional fragment.
33. The method of paragraphs 29-32, wherein the STIM1 fragment or derivative thereof is a functional fragment.
34. The method of paragraphs 29-33, wherein the STIM1 is STIM1 (233-685) or STIM (233-498).
35. The method of paragraphs 29-34, wherein performing step b) is in the presence or absence of one or more mammalian proteins known to modulate STIM1 regulation of intracellular calcium.

### EXAMPLES

### Example 1

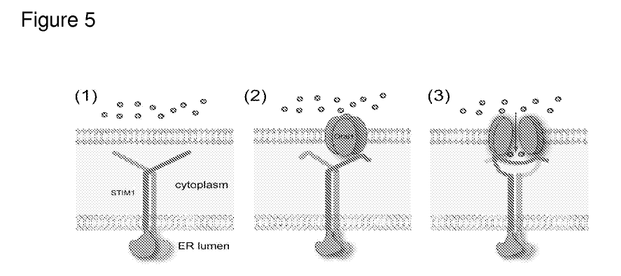

Ca²⁺ influx through the CRAC channel in mammalian T cells and mast cells is essential for transcriptional responses and other effector responses to physiological stimuli (Feske, S. et al., Nat Rev Immunol 7, 690-702 (2007); Oh-hora, M. & Rao, A. Curr Opin Immunol 20, 250-258 (2008); Baba, Y., et al., Nat Immunol 9, 8 1-88 (2008); Vig, M., et al., Nat Immunol 9, 89-96 (2008)). STIM1, a protein anchored in the endoplasmic reticulum (ER), senses depletion of ER Ca²⁺ stores (Roos, J., et al., J Cell Biol 169, 435-445 (2005); Liou, J., et al., Curr Biol 15, 1235-1241 (2005); Zhang, S. L., et al., Nature 437, 902-905 (2005)) and gates a plasma membrane Ca²⁺ channel whose pore subunit is ORAI1 (Feske, S., et al., Nature 441, 179-185 (2006); Zhang, S. L., et al., Proc Natl Acad Sci USA 103, 9357-9362 (2006); Vig, M., et al., Science 312, 1220-1223 (2006); Yeromin, A. V., et al., Nature 443, 226-229 (2006); Prakriya, M., et al., Nature 443, 230-233 (2006); Vig, M., et al., Curr Biol 16, 2073-2079 (2006)). Recent work has established that the STIM-ORAI pathway is widespread in other mammalian cells and in multicellular organisms across the spectrum from vertebrates to insects to roundworms (Stiber, J., et al., Nat Cell Biol 10, 688-697 (2008); Lyfenko, A. D. & Dirksen, R. T. J Physiol 586, 4815-4824 (2008); Koh, S., et al., Dev Biol 330, 368-376 (2009); Eid, J. P., et al., BMC Dev Biol 8, 104 (2008); Lorin-Nebel, C., et al., J Physiol 580, 67-85 (2007)). In order to dissect the essential steps in STIM-ORAI signalling, ORAI1 was expressed in a sec6-4 strain of the yeast Saccharomyces cerevisiae, which allows isolation of sealed membrane vesicles carrying ORAI1 from the Golgi compartment to the plasma membrane. S cerevisiae itself has no significant reservoir of Ca²⁺ in the ER (Strayle, J., et al., EMBO J 18, 4733-4743 (1999)), does not possess orthologues of the ER Ca²⁺-ATPase or IP₃ receptor (Locke, E. G., et al., Mol Cell Biol 20, 6686-6694 (2000); Silverman-Gavrila, L. B. & Lew, R. R. J Cell Sci 115, 5013-5025 (2002)), and has no STIM or ORAI homologues. It is shown herein by in vitro Ca²⁺ flux assays that bacterially-expressed recombinant STIM1 opens wildtype ORAI1 channels, but not channels assembled from the ORAI1 pore mutant E106Q or the ORAI1 SCID mutant R91W. These experiments demonstrate that the STIM-ORAI interaction is sufficient to gate recombinant human ORAI1 channels in the absence of other proteins of the human ORAI1 channel complex. The experimental evidence for a STIM coiled coil and for direct gating of the ORAI channel supports a simple model (FIG. 5) in which activated STIM1 bridges the gap between ER and plasma membrane, recruits ORAI1 channels through a first interaction with the ORAI C terminus, and opens the channels through interaction with ORAI at a second site.

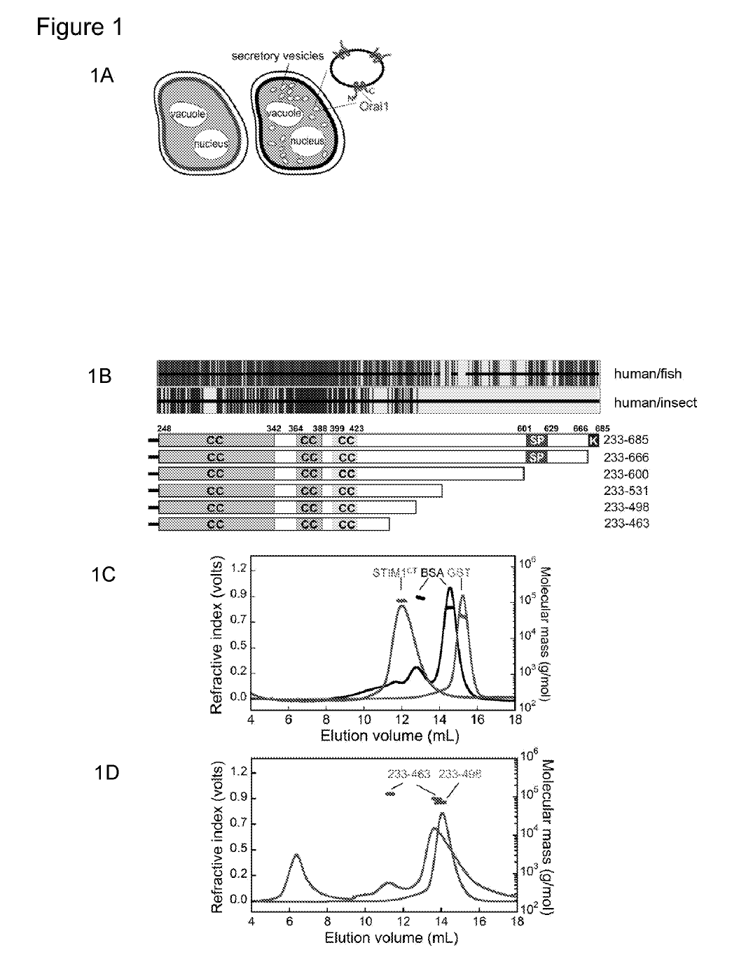

The experiments set out to express properly assembled recombinant ORAI1 channels in the yeast S cerevisiae. The temperature-sensitive sec6-4 mutation of S. cerevisiae disables fusion of vesicles trafficking from the Golgi compartment to the plasma membrane at the restrictive temperature, 37° C., and newly synthesized plasma membrane proteins accumulate in vesicles in the cell cytoplasm (Novick, P., et al., Cell 21, 205-215 (1980); TerBush, D. R., et al., EMBO J 15, 6483-6494 (1996)) (see FIG. 1A). The isolated vesicles have been used in flux assays to investigate transport by several plasma membrane proteins (Nakamoto, R. K., et al., J Biol Chem 266, 7940-7949 (1991); Ruetz, S. & Gros, P. Cell 77, 1071-1081 (1994); Laizé, V., et al., FEBS Lett 373, 269-274 (1995); Coury, L. A., et al., Am J Physiol 274, F34-F42 (1998)), and seemed particularly suited to the planned experiments because recombinant ORAI would be oriented with its cytoplasmic face accessible for interaction with recombinant STIM (FIG. 1A). Immunocytochemistry of Myc-ORAI1 was performed in sec6-4 cells at 25° C. and at 37° C. It was observed that Myc-tagged human ORAI 1 expressed in a sec6-4 strain of S cerevisiae was correctly targeted to the plasma membrane at the permissive temperature 25° C., as indicated by a circumferential pattern of immunocytochemical staining of the Myc tag in most cells, and was retained in the cell interior at the restrictive temperature 37° C. (data not shown). Western blot analysis was used to further verify these results. Western blot analysis was performed to detect Myc-ORAI1 in vesicles isolated from control yeast or from yeast expressing wildtype ORAI, ORAI(R91W), or ORAI(E106Q). Ponceau S staining after transfer to nitrocellulose was performed to verify appropriate transfer, followed by and staining with monoclonal anti-Myc antibody. The results indicated that secretory vesicles isolated according to a standard protocol from cells that had been incubated at the restrictive temperature contained Myc-ORAI1 detectable by Western blotting (data not shown). All three ORAI proteins were detected in similar amounts.

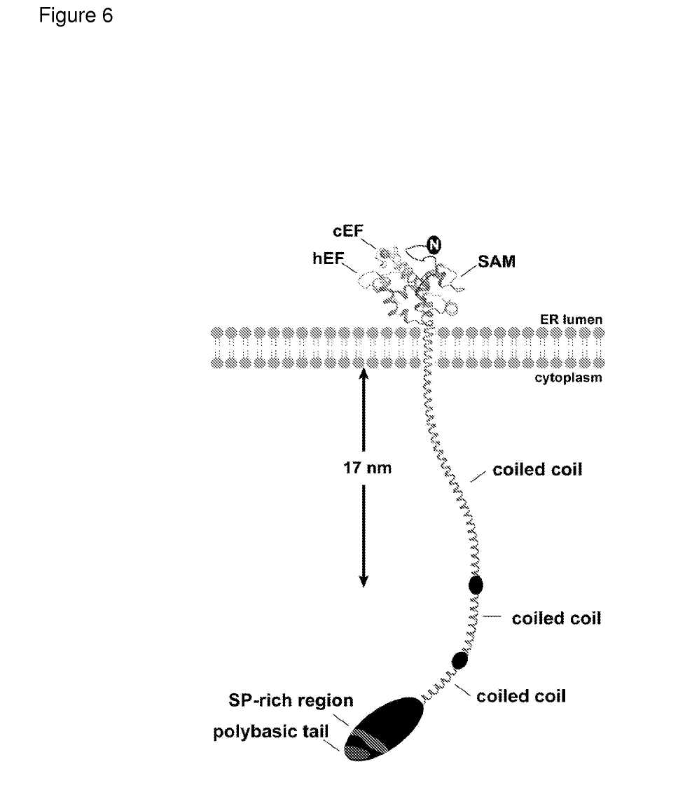

To investigate the interaction by which STIM1 gates ORAI1 channels, experiments were focused on the cytoplasmic region of STIM1, STIM^{CT}, which is sufficient in cells to trigger activation of ORAI1 (Muik, M., et al., J Biol Chem 283, 8014-8022 (2008); Huang, G. N., et al., Nat Cell Biol 8, 1003-10 10 (2006); Zhang, S. L., et al., J Biol Chem 283, 17662-17671 (2008); Penna, A., et al., Nature 456, 116-120 (2008); Ji, W., et al., Proc Natl Acad Sci USA 105, 13668-13673 (2008)). Sequence alignments of vertebrate STIM1 orthologues show the region of pronounced conservation ending around residue 531; additional alignments with insect Stim proteins and with STIM2 show a shorter region of conservation, ending around residue 498 (FIG. 1B). On the premise that interaction either with ORAI itself or with other proteins of the ORAI channel complex is a basic function of STIM proteins that will be reflected in sequence conservation, STIM 1 C-terminal proteins truncated at these sites and at other sites suggested by sequence conservation were expressed and purified (FIG. 1B). The anchoring of STIM in ER and the measured ER-plasma membrane distance (Wu, M. M., et al., J Cell Biol 174, 803-813 (2006)) (FIG. 6) render it unlikely that the initial part of the STIM 1 coiled coil interacts directly with ORAI, but the entire coiled coil was retained in the constructs because of its possible role in proper STIM multimer assembly.

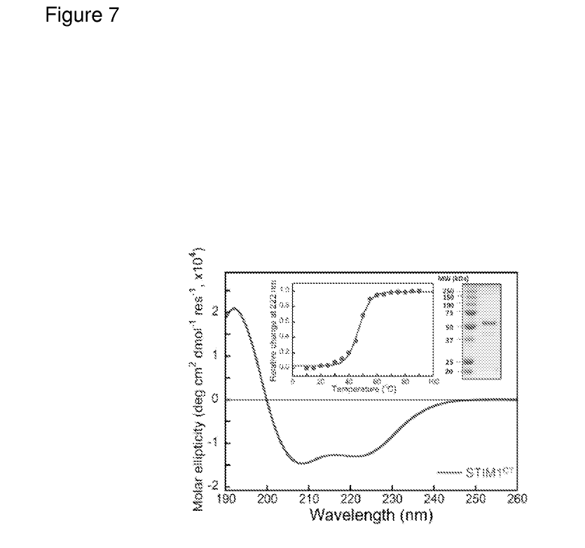

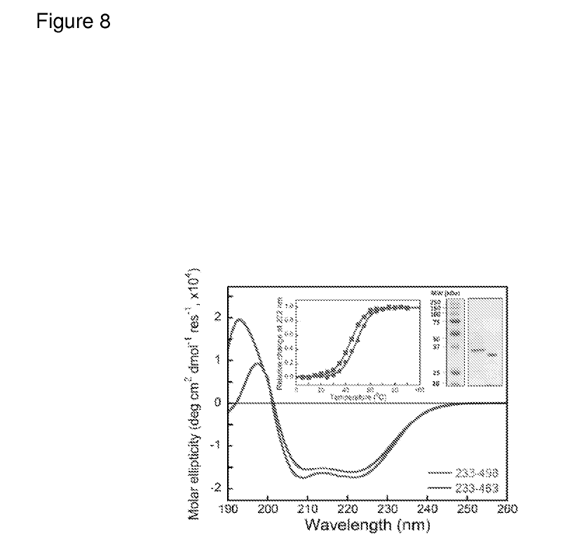

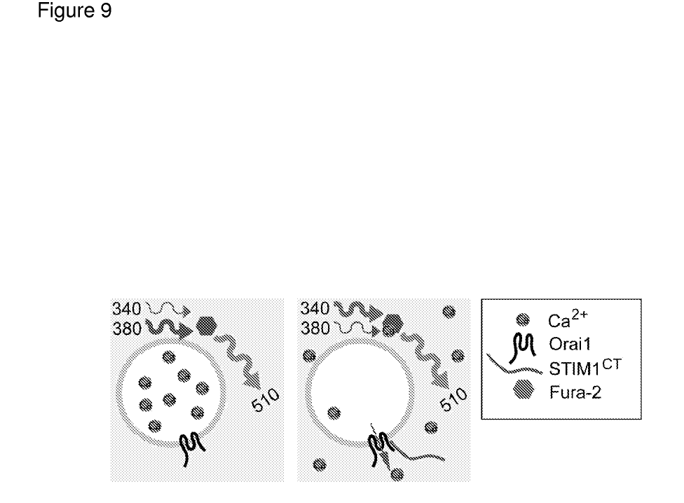

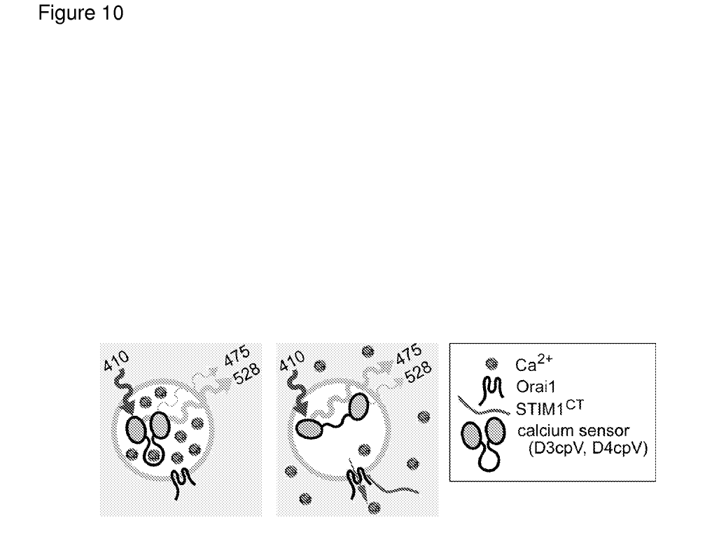

Analysis of recombinant STIM^{CT} by size exclusion chromatography coupled with multi-angle laser light scattering (SEC-MALLS) showed that the purified protein is dimeric under these in vitro conditions (FIG. 1C). STIM1 (233-498) also formed dimers; STIM1 (233-463) was a heterogeneous mixture of trimers and smaller material, tetramers, and large aggregates (FIG. 1D). Circular dichroism (CD) spectroscopy of STIM^{CT}, STIM 1 (233-498), and STIM 1 (233-463) indicated α-helix content, respectively, of 49%, 54%, and 57% (FIGS. 7 and 8). The α-helix content of the latter fragments and the [θ]222/[θ]208 ratio ˜1 provide strong experimental support for the predicted STIM coiled coil.

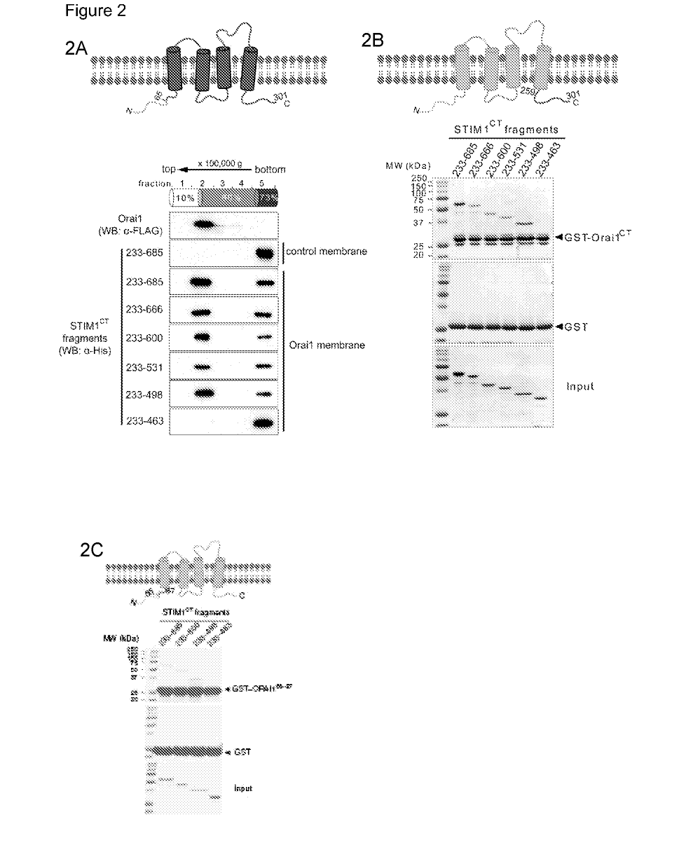

A membrane flotation assay was used to evaluate binding of these recombinant STIM 1 proteins to ORAI1. For these experiments ORAI1 (65-301) was expressed, an N-terminally truncated ORAI1 protein that forms functional Ca²⁺ channels in mammalian cells (Li, Z., et al., J Biol Chem 282, 29448-29456 (2007)), in the yeast Pichia pastoris. Microsomal membranes were prepared from P pastoris expressing ORAI1 (65-301), layered at the bottom of a discontinuous sucrose gradient, and centrifuged. After centrifugation, the recombinant ORAI 1 was recovered near the top of the gradient in the fraction visually identified as containing membranes (FIG. 2A), as expected for an integral membrane protein. STIM1^{CT} centrifuged together with control membranes from yeast not expressing ORAI1 remained at the bottom of the gradient, as expected for a soluble protein. However, when STIM1^{CT} was mixed with membranes containing ORAI1 and then centrifuged, a substantial fraction of STIM 1^{CT} rose with the membranes into the upper part of the gradient, demonstrating its interaction with ORAI1. STIM1 (233-498) and all the longer STIM fragments tested in this assay also clearly bound ORAI, whereas STIM 1 (233-463) did not bind detectably.

Recruitment of ORAI1 to puncta by full-length STIM 1 depends on a direct or indirect interaction of STIM 1 with the C-terminal cytoplasmic tail of ORAI134,51. Therefore the direct interaction of recombinant STIM 1 proteins was next examined with a bacterially expressed fusion protein, GST-ORAI^{CT}, containing the cytoplasmic tail of ORAI1, residues 259-301. STIM 1^{CT} and the other STIM 1 fragments, except for STIM 1 (233-463), bound to GST-ORAI1^{CT} immobilized on resin (FIG. 2B). Binding was dependent on recognition of ORAI^{CT}, since there was no binding to GST alone. Because all input and bound proteins on the gel was visualized with Coomassie Brilliant Blue staining and detected no other proteins, the current experiment is unambiguous proof of a direct protein-protein interaction between STIM 1^{CT} and ORAI1^{CT}.

A conserved region of ORAI1 immediately preceding, and extending into, ORAI1 transmembrane segment 1 is implicated in channel opening (Feske et al., Nature 441: 179-185 (2006); Li et al., J. Biol. Chem. 282: 29448-29456 (2007); Park et al., Cell 136: 876-890 (2009); Derler et al., J. Biol. Chem. 284: 15903-15915 (2009)), and GFP-ORAI1 (48-91) expressed in mammalian cells co-immunoprecipitates with STIM1 (342-448) (Park et al., Cell 136: 876-890 (2009)). Here again, the protein-protein interaction is direct, because purified recombinant STIM1CT bound to purified GST-ORAI1 (65-87) (FIG. 2C). This experiment required a large amount of input protein, and the fraction of input STIM1 retained by immobilized GST-ORAI1 peptide was small, indicating that the interaction is weaker than the STIM1^{CT}-ORAI1^{CT} interaction.

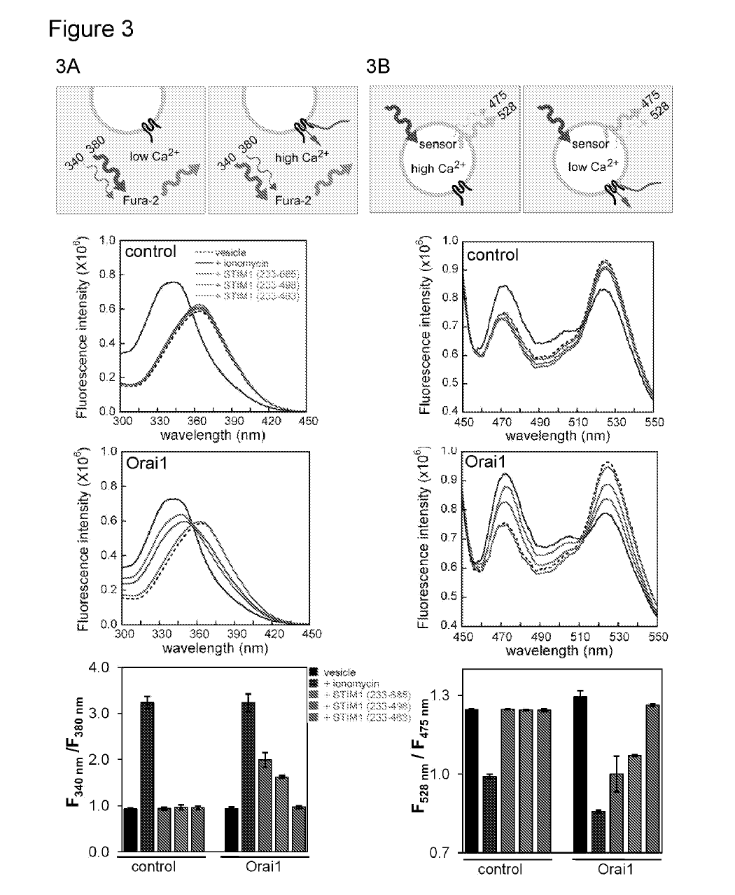

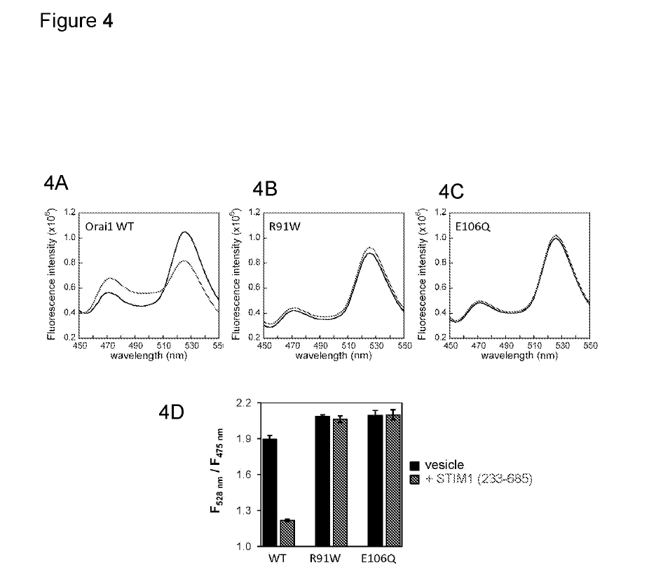

It was next asked whether STIM-ORAI protein-protein interaction is sufficient to open the ORAI Ca²⁺ channel. Vesicles obtained from S cerevisiae expressing ORAI were incubated under conditions where Fura-2 was the principal Ca²⁺ buffer in the extravesicular solution in order to monitor Ca²⁺ efflux (Meyer, T., et al., Biochemistry 29, 32-37 (1990)). Treatment with the Ca²⁺ ionophore ionomycin increased the prominence of the peak of the Fura-2 excitation spectrum near 340 nm (Ca²⁺-dye complex) relative to the peak near 365 nm (free dye), indicating release of Ca²⁺ to the extravesicular solution (FIG. 3A). Addition of STIM1^{CT} or STIM1 (233-498) also elicited efflux of Ca²⁺ from vesicles with ORAI, whereas STIM1 (233-463) had little effect (FIG. 3A). The effect of STIM1^{CT} and STIM 1 (233-498) required ORAI 1, because neither STIM 1 fragment was effective in releasing Ca²⁺ from control vesicles lacking ORAI1 (FIG. 3A).

In complementary measurements, vesicular Ca²⁺ was monitored directly by coexpressing ORAI1 with the Ca²⁺ sensor D3 cpV or D4 cpV53 fused to the α-mating factor secretion signal to target the sensor to the vesicles. Vesicles that had incorporated ORAI1 were isolated from sec6-4 yeast incubated at the restrictive temperature and were diluted into assay buffer. The intravesicular sensor gave a stable FRET signal at ˜528 nm due to the internal Ca²⁺, indicating that the vesicles were not leaky to Ca²⁺. Treatment with ionomycin reduced the FRET signal at ˜528 nm and increased the donor signal at ˜475 nm, corresponding to depletion of vesicular Ca²⁺ (FIG. 3B). Addition of either STIM1^{CT} or STIM1 (233-498) likewise triggered substantial loss of vesicular Ca²⁺ (FIG. 3B). With control vesicles lacking ORAI1, only ionomycin was effective in releasing Ca²⁺ (FIG. 3B).

Confirmation that Ca²⁺ release from the vesicles was due to gating of the ORAI 1 channel was sought. Two mutant ORAI proteins that do not support Ca²⁺ influx in mammalian cells ORAI1 (R91W), which cannot be activated by STIM 1 (Feske, S., et al., (2006), supra), and ORAI1 (E106Q), which is disabled in the ion-conducting pore (Prakriya, M., et al., (2006), supra) were expressed individually in the sec6-4 strain for control experiments. Although each mutant ORAI protein was present in isolated vesicles at levels comparable to wildtype ORAI (as determined by Western blot analysis), STIM^{CT} did not elicit Ca²⁺ release from vesicles containing either mutant protein. It was concluded that the in vitro assay reflects STIM 1-dependent gating of ORAI1 and Ca²⁺ flux through the ORAI1 channel pore.

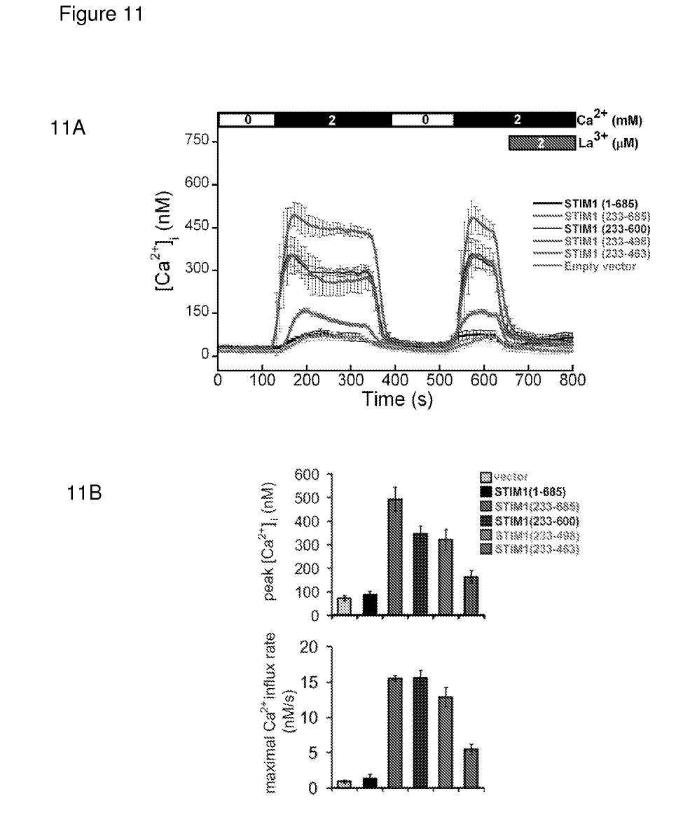

To determine the competence of STIM 1 fragments to activate ORAI1 in mammalian cells, Myc-tagged STIM 1^{CT} and its truncated variants were introduced by retroviral expression into STIM1−/− T cells, and their ability to elevate resting Ca²⁺ permeability was examined in cells loaded with Fura-2. As in human Jurkat T cells (Huang, G. N., et al., (2006); supra), expression of the soluble cytoplasmic portion of STIM 1 activated Ca²⁺ influx into STIM1−/− murine T cells (FIG. 11). Elevated Ca²⁺ permeability was evident in a marked increase in cytoplasmic [Ca²⁺] on changing to medium containing 2 mM Ca²⁺ after brief exposure to nominally Ca²⁺-free medium. The steepest rise of cytoplasmic [Ca²⁺] in cells expressing STIM 1^{CT} reached ˜15 nM/s, which is comparable to the maximum rate in wildtype murine T cells activated by Ca²⁺ store depletion (Oh-hora, M., et al., Nat Immunol 9, 432-443 (2008)) and indicates that the STIM fragments are efficient in activating endogenous CRAC channels. The truncated STIM1 proteins STIM1 (233-600) and STIM1 (233-498) activated a prominent constitutive Ca²⁺ influx (FIG. 11). The rate of rise of cytoplasmic Ca²⁺ was nearly comparable to the rate with STIM 1^{CT}, although the maximum levels of cytoplasmic Ca²⁺ were somewhat less than with STIM1^{CT}. STIM 1 (233-463) conferred a smaller but very clear constitutive Ca²⁺ influx. Retroviral vector alone conferred no constitutive influx above the level in control cells, as previously shown (Oh-hora, M., et al., (2008), supra).

The localization of mCherry-STIM1 proteins in transiently-transfected HEK293 cells was investigated. mCherry-STIM1 (233-683), or (233-498) or (233-463) expressed alone, and also mCherry-STIM (233-683), or (233-498) or (233-463) expressed with ORAI-EYFP in the plasmid ratios 1:0, 0.5:1, 1:1, and 2:1 were examined. Micrographs showing the localization of the proteins indicated that STIM1^{CT}, STIM 1 (233-498), and STIM 1 (233-463) all decorated the plasma membrane of HEK293 cells when coexpressed with ORAI1 (data not shown). Little to no decoration was observed in the absence of coexpressed ORAI1. This is consistent with the functional assay in T cells and with recent reports in the literature (Muik, M., et al., J Biol Chem 283, 8014-8022 (2008); Penna, A., et al., Nature 456, 116-120 (2008); Ji, W., et al., Proc Natl Acad Sci USA 105, 13668-13673 (2008); Yuan, J. P., et al., Nat Cell Biol 11, 337-34 3 (2009); Muik, M., et al., J Biol Chem 284, 8421-8426 (2009); Park, C. Y., et al., Cell 136, 876-890 (2009)). The overexpressed STIM proteins were not prominently recruited to the plasma membrane in cells expressing only the low native levels of ORAI1, demonstrating that all three STIM fragments interact with the ORAI1 channel complex in mammalian cells. The contrasting failure to detect binding of STIM 1 (233-463) in vitro might conceivably reflect the state of the bacterially-expressed protein as a mixture of trimers, tetramers, and undefined large aggregates. A more intriguing possibility, however, is that a weak interaction of STIM 1 (233-463) with ORAI is stabilized and rendered productive by additional proteins in mammalian cells.

The advantage of the absence of STIM-ORAI signaling in the yeast Saccharomyces cerevisiae allows one to show that recombinant STIM 1 gates ORAI1 directly, without assistance from other proteins with a dedicated role in the mammalian store-operated Ca²⁺ entry pathway. Other cellular proteins might modulate store-operated Ca²⁺ influx, but they are not essential to channel function. The work described herein indicates that native STIM1 in cells interacts directly with ORAI1 across the ˜17-nm distance (Wu, M. M., et al., J Cell Biol 174, 803-813 (2006)) that separates the ER and plasma membrane. The store-operated channels formed by ORAI1 (65-301) and ORAI1 (74-301) have short cytoplasmic portions that cannot span this distance. Rather, it is shown herein that STIM1 forms a coiled coil of sufficient length to position the central region of the STIM1 cytoplasmic domain near the cytoplasmic face of the plasma membrane (Park et al., Cell 136: 876-890 (2009); Yuan et al., Nat. Cell Biol. 11: 337-343 (2009); Muik et al., J. Biol. Chem. 284: 8421-8426 (2009); Kawasaki et al., Biochem. Biophys. Res. Commun. 385: 49-54 (2009)). The latter findings complements evidence that the region of STIM1 encompassing residues 344-442 causes constitutive activation of ORAI1 channels when expressed in mammalian cells. The development of in vitro functional assays, with defined protein reagents, to probe STIM-ORAI interaction and Ca²⁺ flux through the ORAI channel is an essential step toward the rigorous biochemical characterization of STIM-ORAI gating.

### Methods Summary

STIM 1, ORAI 1, and D3 cpV/D4 cpV expression constructs were engineered as described herein. Recombinant STIM 1 fragments were expressed in E coli, purified, and characterized by CD spectroscopy and SEC-MALLS. Binding of STIM 1 fragments to ORAI1 channels in their normal bilayer environment was assessed in a flotation assay, using microsomal membranes prepared from the yeast P pastoris and containing human ORAI1 (65-301). Binding of STIM1 fragments to recombinant ORAI^{CT} was assessed in a pulldown assay using GST-tagged ORAI1^{CT}.

Vesicles carrying ORAI 1 were isolated from S cerevisiae strain NY1 7 according to a standard protocol (Coury, L. A., et al., Methods Enzymol 306, 169-186 (1999); Nakamoto, R. K., et al., J Biol Chem 266, 7940-7949 (1991)). Efflux of Ca²⁺ elicited by STIM1^{CT} fragments was detected by suspending the vesicles in a buffer containing Fura-2 as principal Ca²⁺ buffer, and monitoring the Fura-2 fluorescence excitation spectrum (Meyer, T., et al., Biochemistry 29, 32-37 (1990)). As a complementary approach, ORAI 1 was coexpressed with a Ca²⁺ sensor cameleon D3 cpV or cameleon D4 cpV53 that was targeted to the vesicles, and changes in intravesicular [Ca²⁺] were detected as changes in FRET between CFP and YFP of the sensor.

STIM 1 fragments were transiently expressed in STIM1−/− T cells to assay their ability to reconstitute store-operated Ca²⁺ entry, and corresponding mCherry-STIM1 fragments were transiently expressed in HEK293 cells to examine their localization by confocal fluorescence microscopy.

Accession Numbers.

Predicted STIM protein sequences were obtained from the Ensembl database: zebrafish (Danio rerio), EN SDARP00000080300; stickleback (Gasterosteus aculeatus), ENSGACP000000 15080; medaka (Oryzias latipes), ENSORLP00000005059; Japanese pufferfish (Takifugu rubripes), ENSTRUP00000007672; spotted green pufferfish (Tetraodon nigroviridis), EN STN I P00000019346; mosquito (Aedes aegypti), AAEL0 13609-PA; mosquito (Anopheles gambiae), AGAP000175-PA; and fruitfly (Drosophila melanogaster), FBpp0073955.

Plasmids.

The cDNAs encoding mouse STIM 1^{CT} fragments (residues 233-685, 233-666, 233-600, 233-531, 233-498, or 233-463) were amplified via PCR and cloned into the pProEX HTb vector (Invitrogen) between the BamHI and XhoI sites for expression as His-tagged proteins. For production of the C-terminal cytoplasmic tail of ORAI 1 as a GST fusion protein, coupled to GST through the flexible linker—GSGSRGSPEF—(SEQ ID NO: 1), cDNA encoding ORAI1 residues 259-301 was amplified using PCR and cloned into the pGEX-4T-1 vector (GE Healthcare) between the BamHI and NotI sites. A plasmid encoding ORAI1 (65-87) as a GST fusion protein with the linker—GSGGGS—(SEQ ID NO: 9) was produced by inserting the corresponding Cdna into the pGEX-2T vector (GE Healthcare).

Retroviral expression plasmids encoding mouse STIM 1 and its cytoplasmic fragments (residues 233-685, 233-600, 233-498, or 233-463) were constructed by inserting the corresponding coding sequences between the XhoI and EcoRI sites of the vector pMSCV-CITE-eGFP-PGK-Puro (Clontech), which allows for coexpression of the inserted gene, GFP, and a puromycin resistance gene. GFP expression was used to estimate transducing efficiency.

An ORAI1-YFP expression plasmid was constructed by cloning the human ORAI1 cDNA (accession number NM_{—}032790) into the vector pEYFP-N1 between the EcoRI and AgeI sites, in frame with the YFP coding sequence. mCherry-tagged STIM 1^{CT} fragments were made by subcloning mCherry between the BamHI and EcoRI sites of the vector pcDNA3.1(+) (Invitrogen), and subsequently inserting cDNA encoding STIM1^{CT} fragments between the EcoRI and XhoI sites.

The yeast expression vector pBEVY-GU60 allows for simultaneous expression of two transgenes by means of the bidirectional GAL1-10 promoter. The FRET-based calcium sensors D3 cpV and D4 cpV53 were fused at the N-terminus with the S. cerevisiae α-mating factor secretion signal (Bitter, G. A., et al., Proc Natl Acad Sci USA 81, 5330-5334 (1984)), which directs proteins into the secretory pathway, and introduced into pBEVY-GU in the following way. Vector pPIC9 (Invitrogen) served as template for PCR amplification of the α-mating factor secretion signal (5′ primer: TTATTAGAATTCCAAACGATGAGATTTCCTTCAATTTT (SEQ ID NO: 2); 3′ primer: CCTTGCTCACAGCTTCAGCCTCTCTTTTCTCGAG (SEQ ID NO: 3)), introducing an EcoRI site at the 5′ end, and a sequence complementary to that encoding the N-terminus of the calcium sensor at the 3′ end. DNA encoding the N-terminal portion of the calcium sensor D3 cpV, comprising CFP, was amplified by PCR (5′ primer: GAGGCTGAAGCTGTGAGCAAGGGCGAGGAGC (SEQ ID NO: 4); 3′ primer: ATAATAGCGGCCGCTTAATGCATGCGGGCGGCGGT (SEQ ID NO: 5)), introducing an SphI site followed by a stop codon and a NotI site at the 3′ end of the resulting DNA fragment. PCR-mediated fusion of the fragments representing the α-mating factor secretion signal and the sensor CFP fragment yielded αCFP, which was digested with EcoRI and NotI and subcloned into pSM703. Insertion of the SphI-NotI fragment of D3 cpV into αCFP/pSM703 resulted in the generation of αD3 cpV/pSM703. αD3 cpV was excised with EcoRI and subcloned into pBEVY-GU to create αD3 cpV/pBEVY-GU. Human ORAI1 was Myc-tagged at the N-terminus by PCR (5′ primer: TTATTAGAATTCATCAATATGGAACAAAAATTGATTTCTGAAGAAGATT TGGGTTCTGGTCATCCGGAGCCCGCCCCG (SEQ ID NO: 6); 3′ primer: ATAATAGGATCCCTAGGCATAG TGGCTGCCGGGC (SEQ ID NO: 7)) and subcloned as an EcoRI-BamHI fragment into pSM703. A fragment containing Myc-ORAI1 was excised from the resulting plasmid by EcoRI digestion, blunt-ended with the Klenow fragment of DNA polymerase I, and cut with AvrII. The resulting fragment was subcloned into αD3 cpV/pBEVY-GU that had been digested with BamHI, blunt-ended with Klenow fragment, and subsequently digested with XbaI, resulting in the expression plasmid Myc-ORAI1/αD3 cpV/pBEVY-GU. αD4 cpV/pBEVY-GU and Myc-ORAI1/αD4 cpV/pBEVY-GU were generated by replacing the SphI-BglII fragment of the D3 cpV constructs with the corresponding fragment of D4 cpV. Vectors for simultaneous expression of the calcium sensor D3 cpV and a functionally compromised ORAI protein were produced by exchanging the BspEI-BbvCI fragment from an ORAI1 (R91W) (Feske, S., et al., Nature 441, 179-185 (2006)) or an ORAI1 (E106Q) (Gwack, Y., et al., J Biol Chem 282, 16232-16243 (2007)) plasmid for the corresponding fragment of Myc-ORAI1/αD3 cpV/pBEVY-GU. All constructs involving PCR were verified by sequencing. For protein expression, yeast cells were transfected by electroporation (Becker, D. M. & Guarente, L. Methods Enzymol 194, 182-187 (1991))

Recombinant Protein Expression and Purification.

E coli strain BL21 (DE3) cells were transformed with plasmids encoding STIM 1^{CT} fragments or GST-ORAI^{CT} or GST-ORAI1 (65-87) and grown at 37° C. in LB medium with 100 mg/L of ampicillin. Protein expression was induced when OD₆₀₀ of the culture reached 0.6 by addition of 300 μM of isopropyl-3-D-thiogalactopyranoside (IPTG) followed by incubation for another 3 to 4 hours. Harvested cells were resuspended in buffer containing 50 mM Tris pH 7.5, 150 mM KCl, 1 mM TCEP (tris(2-carboxyethyl)phosphine), and protease inhibitor cocktail (Roche), and sonicated. Cellular debris was removed by centrifugation and the lysate was applied to Ni2+-nitrilotriacetic acid-agarose beads (Qiagen).

Bound recombinant proteins were eluted in 50 mM Tris pH 7.5, 300 mM imidazole, 150 mM KCl, 1 mM TCEP, then further purified by cation exchange on SP-sepharose (GE healthcare) and, in some cases, by gel filtration on a Superdex 200 column (GE healthcare). GST-ORAI1^{CT} and GST-ORAI1 (65-87) were purified using Glutathione Sepharose 4B resin (GE Healthcare) following the manufacturer's protocol. Protein concentrations were determined using the Bradford method.

Circular Dichroism (CD) Spectroscopy.

CD spectra of STIM 1^{CT} fragments were recorded in a Jasco-810 spectropolarimeter at 25° C. using a 1-mm path length quartz cell with the protein concentration at 20 μM in 10 mM Tris-HCl, 150 mM KCl, 1 mM DTT at pH 7.5. All spectra were obtained as the average of at least ten scans with a scan rate of 50 nm/min. The ellipticity was measured from 190 to 260 nm and converted to mean residue molar ellipticity (deg cm² dmol⁻¹ res⁻¹). The calculation of secondary structure elements was performed by using DICHROWEB, an online server for protein secondary structure analyses (Whitmore, L. & Wallace, B. A. Nucleic Acids Res 32, W668-W673 (2004)). Thermal unfolding measurements were performed using a 1 mm quartz cell with a protein concentration of 20 μM in the same buffer. To obtain the thermal transition point, the signal changes at 222 nm were fitted using the equation: ΔS=ΔSₘₐₓ/(1+e^{(Tm-T))/k}), where ΔS and ΔSₘₐₓ are the signal changes at each data point and at the final data point, Tₘ and T are the transition temperature and experimental temperature, respectively, and k represents the transition rate.

Size Exclusion Chromatography Coupled with Multiple Angle Laser Light Scattering (SEC-MALLS).

SEC-MALLS measurements were carried out on a BioRad FPLC system with a Pharmacia S200 gel filtration column and in-line DAWN-EOS multi-angle light scattering detector (18 detectors) and OptiLab REX refractive index unit (Wyatt Technology). Samples (100 μg protein) were chromatographed in 25 mM Tris-HCl pH 7.5, 150 mM KCl, 1 mM DTT at ambient temperature. GST and bovine serum albumin (BSA) were used as standards to calibrate the system. Molecular weights were calculated by using a protein refractive index increment of 0.187 mL/g. Analysis of the data was performed with ASTRA 5 software (Wyatt Technology).

Expression of ORAI Proteins in P. Pastoris.

Human ORAI1 (65-301) with an N-terminal FLAG tag was subcloned into the pPIC3.5K vector (Invitrogen) for expression of ORAI1 in the methylotrophic yeast Pichia pastoris. Separate cDNAs, one based on the wildtype human coding sequence and one a synthetic cDNA (DNA2.0) with codons optimized to boost protein production in yeast, were subcloned for initial experiments. In some constructs, the cDNA encoded the mutation E106Q to disable ORAI pore function or N223A to prevent N-glycosylation of the protein or both.

P pastoris strain GS115 was transformed with SalI-linearized pPIC3.5K-ORAI1 by electroporation and selected on plates lacking histidine. H is transformants were further grown in the presence of 0.25-4 mg/mL G418 to select for yeast that had integrated multiple copies of pPIC3.5K into the genome. Selected colonies capable of growth on 0.5-4 mg/mL G418 were grown in small-scale culture, induced with methanol as detailed below, and their level of ORAI expression determined by Western blotting for the FLAG tag. P pastoris transformants were grown in minimal dextrose medium lacking histidine and induced with 0.5% methanol for 6-18 h. Cells were pelleted at 3,000×g for 15 min at 4° C. and resuspended in breaking buffer containing 50 mM sodium phosphate, 1 mM TCEP, 1 mM EDTA, 5% glycerol and complete protease inhibitor cocktail (Roche). An equal amount of glass beads (0.5 mm, BioSpec) was added to the cell suspension and cells were disrupted using either a vortex mixer or a BeadBeater (BioSpec) with a Teflon rotor on ice. The cell lysate was centrifuged at 3,000×g for 5 min at 4° C. to remove cell debris, nuclei, and glass beads. The postnuclear supernatant was centrifuged at 100,000×g for 1 h at 4° C. using either a Beckman airfuge or an ultracentrifuge, depending on the volume of material being processed. To further remove ribosomal components, the pelleted membranes were resuspended in membrane resuspension buffer (10 mM HEPES pH 7.4, 150 mM NaCl, 1 mM TCEP, 10% glycerol), briefly sonicated, mixed with 25 mM EDTA, layered over a sucrose cushion, and centrifuged at 100,000×g for 1 h at 4° C. The pelleted membranes were again resuspended in membrane resuspension buffer, and stored at −80° C. until use.

Pichia Membrane Flotation Assay.

Microsomal membranes, prepared from yeast Pichia pastoris expressing human ORAI1 (65-301) with an N-terminal FLAG tag and the substitutions E106Q and N223A, were resuspended in 10 mM HEPES pH 7.4, 150 mM NaCl, 1 mM DTT, 10% glycerol, briefly sonicated, brought to 25 mM EDTA by addition of EDTA from a concentrated stock, layered over a 0.5 M sucrose cushion, and centrifuged at 149,000×g for 1 h at 4° C. in a Beckman airfuge. The pelleted membranes were resuspended in the same buffer without EDTA and stored at −80° C. For sucrose gradient membrane flotation, 30 μL of resuspended membrane and 10 μL of 5-10 μM purified STIM1^{CT} fragment were mixed with 400 μL of 73% (w/v) sucrose and loaded at the bottom of a 2-mL centrifuge tube. After incubation on ice for 30 min, the mixture at the bottom was overlaid with 1.2 mL of 60% sucrose and further overlaid with 400 μL of 10% sucrose. The solution was centrifuged at 100,000×g for 16 h at 4° C. in a SW60 Ti rotor (Beckman Coulter). Five 400-μL aliquots were collected from the top to bottom. A 40-μL aliquot of each fraction was subjected to electrophoresis in a 4-12% Bis-Tris NuPAGE gel (Invitrogen) and Western blotting. FLAG-tagged ORAI1 was detected using anti-FLAG M2 monoclonal antibody (Sigma) and His-tagged STIM1^{CT} fragments were detected using anti-His-Tag monoclonal antibody (27E8 clone, Cell Signaling Technology).

GST Pull Down Assay.

GST-ORAI1^{CT} (50-60 μg) or GST-ORAI1 (65-87) (120 μg), or GST was immobilized on glutathione-Sepharose 4 Fast Flow resin (GE Healthcare) and incubated with each purified STIM1^{CT} fragment (100-150 μg) in 100 μl phosphate-buffered saline pH 7.4 (PBS) supplemented with 1 mM DTT and complete protease inhibitor cocktail (Roche) for 1 h at 4° C., and then washed eight times with 1 ml PBS to eliminate nonspecific binding. To avert formation of SDS-resistant aggregates that sometimes formed upon boiling, the beads were incubated at 65° C. with an equal volume of 2×SDS gel loading buffer, briefly centrifuged, and subjected to SDS-PAGE on a 15% polyacrylamide gel. Gels were stained with Coomassie Brilliant Blue R-250 for protein visualization.

Preparation of sec6-4 Secretory Vesicles.

S cerevisiae strain NY17 featuring the temperature-sensitive sec6-4 mutation was used for protein expression and purification of secretory vesicles as described (Nakamoto, R. K., et al., J Biol Chem 266, 7940-7949 (1991); Coury, L. A., et al., Methods Enzymol 306, 169-186 (1999)). Cells were grown to midlog phase at 25° C. in uracil-deficient synthetic complete medium containing glucose, which was subsequently exchanged for galactose to induce transgene expression over a period of 8 hours. The temperature was then switched to 37° C. for 3 hours to force intracellular accumulation of secretory vesicles. Cells were collected by centrifugation at 4,000×g for 5 min at 4° C., washed once in ice-cold water, resuspended at a concentration of 50-60 OD₆₀₀ units/mL in 10 mM DTT, 100 mM Tris pH 9.4, and shaken gently at room temperature for 10 min. Cells were pelleted and resuspended at 50-60 OD600 units/mL in spheroplast buffer (1.4 M sorbitol, 50 mM K₂HPO₄ pH 7.5, 10 mM NaN₃, 40 mM β-mercaptoethanol) containing Zymolyase 20T (50 units per g of yeast wet weight) and incubated for up to 1 h at 37° C. Spheroplasts were harvested at 3,000×g for 5 min at 4° C., resuspended in spheroplast buffer containing 10 mM MgCl₂ and 1 mg/ml Concanavalin A at a density corresponding to 50 OD₆₀₀ units/mL of the starting culture, and gently shaken at 4° C. for 15 min. Lectin-coated spheroplasts were pelleted in a Sorvall H6000A rotor at 3000×g for 5 min at 4° C., and resuspended at 60-70 OD₆₀₀ units/mL in storage buffer (0.8 M sorbitol, 10 mM triethanolamine acetate pH 7.2, containing complete protease inhibitor cocktail EDTA-free (Roche)) and homogenized at 4° C. in a Dounce homogenizer with 30 strokes of the pestle. Unlysed cells, cell debris, mitochondria, and nuclei were pelleted by centrifugation at 20,000×g for 10 min at 4° C. in an SW 55 Ti rotor. Vesicles in the supernatant were pelleted by centrifugation at 144,000×g for 1 h at 4° C. in an SW 55 Ti rotor, resuspended in storage buffer, and stored at 4° C. Myc-tagged ORAI1 was detected in Western blots using the murine anti-Myc monoclonal antibody 9E10.

Fluorescence-Based Ca2+ Flux Assays Using sec6-4 Secretory Vesicles.

For measurements with Fura-2, all reagents and buffers, including ionomycin, Fura-2 pentasodium salt (Invitrogen) and purified STIM 1^{CT} fragments, were pretreated with Calcium Sponge S resin (Invitrogen) to remove traces of Ca²⁺. The effectiveness of this treatment was verified by examining Fura-2 spectra of the working solutions. To load Ca²⁺ into the secretory vesicles, sec6-4 vesicles were incubated with 5 mM CaCl₂ in storage buffer, briefly sonicated and centrifuged at 100,000×g for 15 min at 4° C. in a Beckman airfuge. The pelleted vesicles were gently resuspended in 20 mM Tris pH 7.4, 100 mM KCl, and passed through Calcium Sponge S resin in a Micro-spin column (˜30 μm pore size; Pierce) to reduce extravesicular residual metal ions to low nanomolar levels (Meyer, T., et al., Biochemistry 29, 32-37 (1990)).

Fura-2 spectra were recorded at ambient temperature, 22-25° C., in 20 mM Tris pH 7.4, 100 mM KCl, containing 10 μM metal-free Fura-2. Excitation spectra were recorded by scanning from 300-450 nm while monitoring emission at 510 nm, using a 2-4 nm slitwidth for both excitation and emission. Where indicated, purified STIM1^{CT} fragment was added to the cuvette to a final concentration of 2-3 μM, or ionomycin was added to a final concentration of 20 μM.

Changes in intravesicular [Ca²⁺] were monitored with the modified Ca²⁺ sensors cameleon D3 cpV and D4 cpV53. Measurements were made at ambient temperature, 22-25° C., in 20 mM Tris pH 7.4, 100 mM KCl. Emission spectra were recorded from 450-560 nm with excitation set at 410 nm, with a 5-8 nm slitwidth for both excitation and emission, and corrected by subtracting the spectrum obtained with buffer alone. A step intended to load the vesicles with Ca²⁺, as described above, appeared to cause only a modest increase in intravesicular [Ca²⁺], but improved the signal in the case of D4 cpV. This step had little effect on the signal from D3 cpV. The appropriate STIM concentration is to some extent dependent on the concentration of vesicles. For the experiments shown, STIM1^{CT} and STIM fragments were used at a final concentration of 2-3 μM, and vesicles at a protein concentration of 25-75 μg/ml.

T Cells Differentiation, Retroviral Transduction and Stimulation.

Primary CD4⁺ cells were purified from spleen and lymph nodes of Stim1^{f1/f1} CD4-Cre⁺ mice using Dynal magnetic beads (Invitrogen) following the manufacturer's instructions. Purified T cells were stimulated with anti-CD3 and anti-CD28, transduced with retroviruses obtained from transfected Phoenix packaging cells, and further expanded in IL-2-containing medium as described (Oh-hora, M., et al., Nat Immunol 9, 432-443 (2008); Ansel, K. M., et al., Nat Immunol 5, 1251-1259 (2004)). The transduction efficiency (>80%) was estimated by evaluating GFP expression using flow cytometry.

Single-Cell [Ca2+]i Measurement.

Retrovirus-transduced CD4⁺ cells isolated from Stim1^{f1/f1} CD4-Cre⁺ mice were incubated overnight in IL-2 free RPMI medium and loaded with 2 μM Fura-2-acetoxylmethyl ester (Invitrogen) for 45 min at ambient temperature in the dark. T cells were further attached to coverslips precoated with 0.01% poly-L-lysine, mounted to a RC-20 closed-bath flow chamber (Warner Instrument), and bathed in Ringer's solution (155 mM NaCl, 4.5 mM KCl, 2 mM CaCl₂, 1 mM MgCl₂, 10 mM D-glucose and 5 mM HEPES, pH 7.4). For nominally Ca²⁺-free Ringer's solution CaCl₂ was replaced with MgCl₂. After 5 minutes of equilibration in Ringer's solution, [Ca²⁺]i measurement commenced with ˜3 min perfusion of T cells with Ca²⁺-free Ringer's solution at ambient temperature, followed by perfusion of Ringer's solution containing 2 mM CaCl₂. The cycle was repeated once with final addition of Ringer's solution supplemented with 2 μM LaCl₃ to block Ca²⁺ flux. Images were acquired using a Zeiss Axiovert S200 epifluorescence microscope and OpenLab imaging analysis software (Improvision). The fluorescence data (F_{340 nm}/F_{380 nm}) were collected every 4 s and analyzed as described (Oh-hora, M., et al., (2008), supra; Feske, S., et al., Nature 441, 179-185 (2006)). In each experiment, for each experimental condition, 50-100 GFP-positive CD4+ T cells were analyzed.

Confocal Fluorescence Imaging.

HEK293 cells were transiently transfected with ORAI-YFP or mCherry-STIM plasmids using Lipofectamine 2000 (Invitrogen) according to the manufacturer's instructions. At 48 h post-transfection, confocal imaging was performed on a Zeiss LSM 510 laser scanning microscope with a 100× oil-immersion objective lens. YFP was excited at 488 nm with an argon laser and mCherry at 543 nm with a HeNe laser. All the experiments were carried out with cells bathed in Hanks' Balanced Salt Solution at 37° C.

### Example 2

### Lanthanide Binding to ORAI1

Tb3+ luminescence resonance energy transfer (Tb3+-LRET) experiments were performed on a FluoroLog®-3 spectrofluorometer (HORIBA Scientific) with a 1-cm lengthpath cuvette at ambient temperature. Emission spectra were collected from 500 to 600 nm with the excitation set at 282 nm, or at 295 nm to minimize the contribution from tyrosine. The slit widths for excitation and emission were set at 4 nm and 8 nm, respectively. A glass filter with cutoff of ˜320 nm was used to circumvent light scattering. Tb3+, diluted from 200 mM stock solution prepared in 20 mM PIPES pH 6.8 to avoid precipitation, was added to final concentration 10-50 μM into the Pichia membrane samples (100-200 μg total membrane protein) in a buffer containing 20 mM PIPES pH 6.8, 150 mM KCl, 1 mM DTT. Spectra from membranes lacking ORAI1, with the same total membrane protein content, served as negative control.

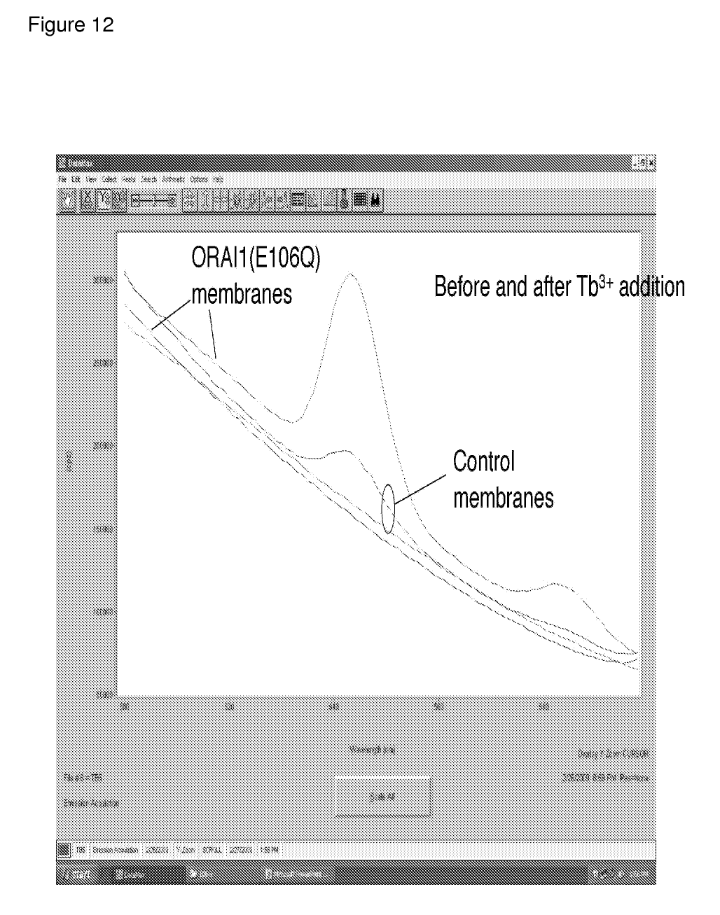

Pichia membranes containing recombinant human ORAI1 (E106Q) and control Pichia membranes. In each case, a scan of luminescence emission from membrane suspension alone was obtained, then Tb3+ was added into the cuvette and a second scan was performed (FIG. 12). Protein content of the two samples was the same. The presence of equivalent amounts of membrane microsomes was also evidenced by identical light scattering in the two cases prior to addition of Tb3+. Characteristic Tb3+ emission peaks at 547 nm and 589 nm were prominent in the ORAI sample, attributable to energy transfer from one or more of the native tryptophan residues of the protein. The amplitude of the Tb3+ signal from control membranes was similar to that obtained by adding the same concentration of Tb3+ to buffer in the absence of membranes.

The lanthanides La3+ and Gd3+ are effective blockers of the CRAC channel pore. It is very likely that luminescent lanthanides such as Tb3+ are reporting on binding in the mouth of the channel. It is possible to raise the signal-to-noise ratio of the assay—known from published studies and from work with other proteins that the scattering signal can be eliminated by using pulsed excitation in a luminometer that is capable of gating out emission during the first 50 microseconds after excitation. A specific Tb3+-LRET signal was also measured with solubilized ORAI1 under suitable conditions.

### Example 3

### ORAI Protein Purified from Pichia pastoris

P. pastoris membranes prepared as described for the flotation assay but without the EDTA stripping step were solubilized by incubation with 4% octyl glucoside; incubated 1-2 hours with Ni-NTA resin with gentle mixing; washed extensively with solubilization buffer; and elute with buffer containing 75-150 mM imidazole. The product was visualized on a silver-stained gel (data not shown) containing the appropriate markers to the left. The lanes containing the fractions of purified ORAI eluted from the Ni-NTA resin indicated that the product was obtained.

### Example 4

### 2-APB Activates Human ORAI3 Expressed in Yeast

Human ORAI3 was expressed in P. pastoris as described below. Then ⁴⁵Ca²⁺ uptake was measured in a suspension of P. pastoris cells expressing ORAI3, after addition of 2-aminoethoxydiphenyl borate (2-APB), a compound known to activate ORAI3 expressed in mammalian cells. For comparison with background ⁴⁵Ca²⁺ uptake, that is uptake in the absence of 2-APB, control suspensions were treated identically, except that the solution added immediately prior to ⁴⁵Ca²⁺ did not contain 2-APB. The results are shown below in Table 1. The rate and extent of ⁴⁵Ca²⁺ uptake were increased by 2-APB. 2-APB did not have this effect on P pastoris cells that were not engineered to express ORAI3, indicating that the increase in uptake is due to the presence of ORAI3, and that ORAI3 assembled into a functional Ca²⁺ channel when expressed in P pastoris.

TABLE 1
Calcium Influx of P. Pastoris expressing human ORAI 3
P pastoris expressing human ORAI3

| ⁴⁵Ca²⁺ uptake (cpm) | | Control | 75 μM 2-APB |
|---|---|---|---|
| ~0.25 | min | 1215.8 | 1913.5 |
| 0.5 | min | 1448 | 2729.9 |
| 1 | min | 2032.2 | 3137.3 |
| 2 | min | 2222.5 | 4662.1 |
| 4 | min | 2923.8 | 6019.2 |

### Methods Summary:

Expression of ORAI3 in P pastoris

A cDNA encoding human ORAI3 (22-295) was subcloned into pPIC3.5K, preceded by a Kozak sequence, initiator methionine codon, and DNA sequence encoding a Myc tag (peptide sequence EQKLISEEDL (SEQ ID NO: 8). The plasmid construct was introduced into P pastoris by electroporation, and transformants selected by growth on selective medium, using the HIS4 gene selection described in the Invitrogen Pichia manual. Transformants growing on selective medium were further selected by growth in the presence of G418 to favor cells that had integrated multiple copies of the plasmid into genomic DNA. All methods for yeast transformation, growth, and selection were as described in the Invitrogen Pichia manual.

Individual colonies were picked, and ORAI3 protein expression was induced by growth for eight hours in MMY medium (containing 0.5% methanol), whose formulation is given below. Cells were collected by centrifugation, and disrupted by vortexing in an Eppendorf tube with 0.5 mm glass beads. Beads, unbroken cells, and debris were removed by centrifugation at 5000 rpm in a benchtop centrifuge. The supernatant from that centrifugation was centrifuged at 100,000×g to pellet cell membranes. Cell membranes were resuspended, protein concentrations determined by Lowry or Bradford assay, and an equal amount of protein from each sample subjected to SDS-polyacrylamide gel electrophoresis and western blotting. The relative levels of Myc-ORAI3 expressed by individual colonies were compared by western blotting with antibody to the Myc tag. Colonies expressing relatively high levels of ORAI3 protein were used for the ⁴⁵Ca²⁺ uptake assay.

### Preparation of Cells for ⁴⁵Ca²⁺ Uptake Assay

Streaked out Pichia glycerol stock on RDB-plate
Grew 2-5 days at 30° C. (until colonies are large enough to easily remove) Seeded 5 mL MGY medium with 1 colony
Grew 2 days in 50 mL Falcon tube (cap partially unscrewed but taped on to allow aeration), 250 rpm, 30° C.
Seeded 50 mL MGY with the 5 mL culture (use a yeast-specific baffled flask; if scaled up, the volume of the culture was 1/10^{th} the volume of the flask)
Grew o/n, 30° C., 250 rpm
At OD ˜2-3, spun down, checked OD and resuspended in MMY at an approximate OD of 6 (i.e., 6×5E7 cells/mL)
Grew 4 h at 28.5° C., 250 rpm, 28.5° C., with the top of the flask covered in gauze (not foil or a lid—a lot of aeration needed for induction. The temperature was under 30° C.)
Measured OD
Washed 3×25 mL in Solution A
Resuspended in Solution A for a final OD of 2 (1×10⁸ cells/mL)

### ⁴⁵Ca²⁺ Uptake Assay

(Prepared everything else: 1 μCi/μL ⁴⁵CaCl₂, 1 mL Solution S aliquots in tubes that can hold at least 2 mL, tips, etc.)
Spun sample (either 1 mL or the multiple-mL timepoint aliquot) down at 2000×g and resuspended in an equal volume of Solution C
(where applicable, added 2-APB or other things such as Solution S or lanthanum here. 75 μM 2-APB with Orai3.)
Added 1 μCi ⁴⁵CaCl₂/Ml
Incubated as appropriate
When timepoint was taken, 1 mL sample (gently pipetted up and down, since Pichia settle) and added to 1 mL Solution S
Collected all samples
(This was repeated for each sample.) Presoaked 2.5 cm Whatman GF/F filter in place on the filter apparatus with 1 mL Solution S
Added the 2 mL sample/Solution S mix to the column
Turned on the vacuum and wash 3×15 mL with Solution W
Let filter dry under vacuum & removed to scintillation vial
Placed filter flat on the bottom of the vial, yeast-side up
Carefully added scintillation fluid
Counted in scintillation counter (⁴⁵Ca settings, if possible: window is 0-750, quench curve calibrations)

```
RDB-Plates
1M sorbitol
2% dextrose
1.34% YNB
4 × 10⁻⁵ % biotin
0.005% amino acids
20 g/L agar
MGY Medium
1.34% YNB
1% glycerol
4 × 10⁻⁵ % biotin
MMY Medium
1.34% YNB
0.5% methanol
4 × 10⁻⁵ % biotin
Solution A
2% dextrose
100 mM MOPS pH 6.8
Solution C
2% dextrose
100 mM MOPS pH 6.8
  1 mM CaCl₂
Solution S
  40 mM MgCl₂
  4 mM LaCl₃
(final concentrations are half that)
Solution W
  10 mM MgCl₂
  5 mM MOPS pH 6.8
```

1. A method of identifying an agent that modulates Ca²⁺ flux through the ORAI channel, comprising:
a) providing S. cerevisiae secretory vesicles functionally expressing ORAI or a functional fragment or derivative thereof;
b) contacting the secretory vesicles with STIM1, or a functional fragment or derivative thereof, and a test agent;
c) monitoring calcium release from the vesicles;
wherein a significant difference in the calcium release from the vesicles compared to a control which lacks the test agent, indicates the test agent modulates Ca²⁺ flux through the ORAI channel.

2. The method of claim 1, wherein the method is performed in the absence of other mammalian proteins.

3. The method of claim 1, wherein the S. cerevisiae is a sec 6-4 strain.

4. The method of claim 1, further comprising contacting the vesicles with another mammalian protein known to modulate STIM1 regulation of intracellular calcium.

5. The method of claim 1, wherein monitoring step c) is with a calcium detection agent that is a fluorescent dye or FRET pairs of GFP variants sensitive to Ca⁺⁺ binding of Fura-2, CFP and/or YFP.

6-10. (canceled)

11. The method of claim 1 wherein the STIM1 functional fragment is STIM1 (233-685), STIM1 (233-498), STIM1 (233-463), or STIM1 (233-600), and wherein the ORAI functional fragment is ORAI1 (65-301), ORAI1 (65-87), or full length ORAI1.

12-13. (canceled)

14. A system comprising:
a) a recombinant ORAI protein or fragment or derivative thereof, expressed in yeast or a vesicle or membrane isolated therefrom.

15. The system of claim 14, further comprising STIM1 protein, or a fragment or derivative thereof.

16-18. (canceled)

19. The system of claim 15, wherein the STIM1 protein, or fragment or derivative thereof, is STIM1 (233-685) STIM1 (233-498), STIM1 (233-463), or STIM1 (233-600), and wherein the ORAI protein or fragment or derivative thereof, is ORAI1 (65-301), ORAI1 (65-87), or full length ORAI1.

20. The system of claim 15, which further comprises another mammalian protein or factor.

21. The system of claim 15, which does not comprise another mammalian protein or factor.

22. A yeast organism, or microsomal membrane or vesicle thereof, that comprises a recombinant, expressed ORAI protein or fragment or derivative thereof.

23. The yeast organism of claim 22 that is genetically engineered to contain, in expressible form, a nucleic acid encoding the ORAI protein or fragment or derivative thereof.

24. The yeast organism, or microsomal membrane or vesicle thereof, of claim 22 that is S. cerevisiae, comprising a recombinant functionally expressed ORAI channel.

25. The yeast organism, or microsomal membrane or vesicle thereof, of claim 22 further comprising a recombinant functionally expressed calcium sensor.

26. (canceled)

27. The yeast organism, or microsomal membrane or vesicle thereof, of claim 22, further comprising another mammalian protein known to modulate STIM1 regulation of intracellular calcium.

28. The yeast organism, or microsomal membrane or vesicle thereof, of claim 22, that is Pichia pastoris.

29. (canceled)

30. A method of identifying an agent that modulates STIM1 binding of a functional ORAI channel, comprising:
a) providing membranes with ORAI or a fragment or derivative thereof incorporated or reconstituted therein; and
b) performing a membrane flotation assay or a binding assay, either of which for binding of STIM1 or a fragment or derivative thereof, to the ORAI in the membrane, in the presence and absence of a test agent;
wherein modulation of binding of STIM1 to the ORAI in the presence of the test agent, compared to binding in the absence of the test agent, indicates that the agent modulates STIM1 binding of a functional ORAI channel.

31. The method of claim 29, wherein ORAI is ORAI1.

32-33. (canceled)

34. The method of claim 30, wherein the STIM1 fragment is STIM1 (233-685) or STIM (233-498).

35. (canceled)

36. The method of claim 30, wherein the membranes are microsomal membranes prepared from P. Pastoris expressing ORAI or a functional fragment or derivative thereof.


SEQUENCE LISTING (10 sequences)

SEQ ID NO: 1 - protein, 10 aa, Artificial Sequence
  Description of Artificial Sequence Synthetic peptide
GSGSRGSPEF

SEQ ID NO: 2 - DNA, 38 bp, Artificial Sequence
  Description of Artificial Sequence Synthetic primer
ttattagaattccaaacgatgagatttccttcaatttt

SEQ ID NO: 3 - DNA, 34 bp, Artificial Sequence
  Description of Artificial Sequence Synthetic primer
ccttgctcacagcttcagcctctcttttctcgag

SEQ ID NO: 4 - DNA, 31 bp, Artificial Sequence
  Description of Artificial Sequence Synthetic primer
gaggctgaagctgtgagcaagggcgaggagc

SEQ ID NO: 5 - DNA, 35 bp, Artificial Sequence
  Description of Artificial Sequence Synthetic primer
ataatagcggccgcttaatgcatgcgggcggcggt

SEQ ID NO: 6 - DNA, 78 bp, Artificial Sequence
  Description of Artificial Sequence Synthetic primer
ttattagaattcatcaatatggaacaaaaattgatttctgaagaagatttgggttctggtcatccggagcccgccccg

SEQ ID NO: 7 - DNA, 34 bp, Artificial Sequence
  Description of Artificial Sequence Synthetic primer
ataataggatccctaggcatagtggctgccgggc

SEQ ID NO: 8 - protein, 10 aa, Artificial Sequence
  Description of Artificial Sequence Synthetic peptide
EQKLISEEDL

SEQ ID NO: 9 - protein, 6 aa, Artificial Sequence
  Description of Artificial Sequence Synthetic peptide
GSGGGS

SEQ ID NO: 10 - protein, 6 aa, Artificial Sequence
  Description of Artificial Sequence Synthetic 6xHis tag
HHHHHH